(19) United States
(12) Patent Application Publication
(10) Pub. No.: US 2010/0135917 A1
(43) Pub. Date: Jun. 3, 2010

(54) COSMETICALLY USED CROSS-LINKED METHYL METHACRYLATE-COPOLYMER

(75) Inventors: Gabi Winter, Shanghai (CN); Marianna Pierobon, Ludwigshafen (DE); Matthias Laubender, Schifferstadt (DE); Son Nguyen Kim, Hemsbach (DE)

Correspondence Address: CONNOLLY BOVE LODGE & HUTZ LLP, 1875 EYE STREET, N.W., SUITE 1100, WASHINGTON, DC, 20006, US

(73) Assignee: BASF Aktiengesellschaft, Ludwigshafen (DE)

(21) Appl. No.: 12/063,374

(22) PCT Filed: Aug. 2, 2006
(86) PCT No.: PCT/EP06/64965

§ 371 Date: Feb. 8, 2008

(30) Foreign Application Priority Data
Aug. 11, 2005 (EP) 05107400.3

(51) Int. Cl. A61K 8/81; A61K 8/73; A61Q 19/10; C08F 220/14; A61Q 5/06; A61Q 5/12; A61Q 1/00; A61Q 5/04; A61Q 5/10; A61Q 9/02; A61Q 15/00; A61Q 17/04

(52) U.S. Cl. 424/47; 424/52; 424/60; 424/65; 424/68; 424/70.5; 424/70.13; 424/70.16; 424/78.31; 510/130; 510/137; 526/318.4

(57) ABSTRACT

The present invention relates to cosmetic preparations which comprise at least one polymer A which comprises, in copolymerized form, methyl methacrylate, methacrylic acid, acrylic acid, if appropriate at least one compound with at least two free-radically polymerizable, olefinically unsaturated double bonds, and if appropriate further olefinically unsaturated compounds, with the proviso that the amount by weight of the methacrylic acid is at least equal to the amount by weight of the acrylic acid.

The present invention relates to cosmetic preparations which comprise at least one polymer A which comprises, in copolymerized form, a) methyl methacrylate, b) methacrylic acid, c) acrylic acid, d) if appropriate at least one compound with at least two free-radically polymerizable, olefinically unsaturated double bonds, and if appropriate further olefinically unsaturated compounds, with the proviso that the amount by weight of component b) is at least equal to the amount by weight of component c).

### PRIOR ART

More stringent environmental requirements and growing ecological awareness increasingly demand ever lower fractions of volatile organic components (VOC) in cosmetic aerosol preparations such as, for example, aerosol hairsprays.

The VOC content in hair sprays is essentially determined by the nonaqueous solvents and the propellants. For this reason, instead of nonaqueous solvents, water is currently being relied on to an increased degree as solvent. However, this replacement of the organic solvents brings with it some problems.

Thus, formulations of the film-forming polymers known from the prior art which meet corresponding VOC requirements are, for example, not sprayable or sprayable only after further dilution and are thus only suitable to a restricted extent for use in hair sprays. Polymer films which are formed from such preparations sometimes do not have the requisite mechanical quality and thus have an inadequate setting effect and poor hold for the hair.

WO 03/062288 and WO 03/061615 describe aqueous hair-setting compositions which comprise an effective amount of a rheology-modifying hair-setting associative polymer of an acid monomer and an associative monomer. Preferred hydrophobic associative monomers are long-chain esters of (meth)acrylic acid. The polymers can comprise further monomers and crosslinkers.

EP-A 0 184 785 describes an aqueous copolymer dispersion of 50-60% by weight of ethyl acrylate, 30-40% by weight of methacrylic acid, 5-15% by weight of acrylic acid and 0.02-0.04% by weight of a polyunsaturated copolymerizable monomer with a solids content of 5-30% by weight which is suitable for thickening aqueous systems, in particular hydrogen peroxide preparations, as are used as developer preparations for oxidation hair dyes and for hair bleaches.

WO 95/05402 describes hair cosmetic preparations which comprise aqueous copolymer dispersions which are obtainable by copolymerization of from 40 to 99% by weight of one or more water-insoluble, monoethylenically unsaturated monomers and 1 to 60% by weight of one or more water-soluble, monoethylenically unsaturated monomers. Optionally, 0 to 30% by weight of one or more ethylenically polyunsaturated monomers can be used.

DE 2 330 957 describes grafted and crosslinked cationic copolymers which are obtained by copolymerization of a) at least one cosmetic monomer, b) dimethylaminoethyl methacrylate, c) polyethylene glycol and d) a polyunsaturated crosslinker.

U.S. Pat. No. 4,543,249 describes nonaerosol compositions which consist to 1-10% by weight of a copolymer of 70-90% by weight of methyl methacrylate and 10-30% by weight of methacrylic acid, where the carboxyl groups of the polymer are neutralized to 50 to 100% with a water-soluble base and the polymer is dissolved in 65-99% strength ethanol.

DE-A 4327514 describes a fine polymer powder, that is rapidly soluble in water, composed of 30-99% by weight of units of alkali metal or preferably ammonium salts of (meth)acrylic acid, which can, if appropriate, be partially replaced by units of free (meth)acrylic acid, and 0.1-5% by weight of crosslinking monomer units and, if appropriate, up to 69.9% by weight of units of hydrophilic comonomers.

### PROBLEM AND SOLUTION

An object of the present invention was to provide polymers for cosmetic, in particular hair cosmetic, preparations which can easily be formulated as pump or aerosol spray in solvents or solvent mixtures with an increased water content, whose formulations are readily sprayable in the form of small uniform droplets and, during and after application, have the lowest possible propensity for foam formation and whose then formed films are not sticky and have good mechanical properties.

Besides the good compatibility with the customary cosmetic-ingredients, the polymers applied to the hair should dry rapidly and impart to the hair good setting and prolonged hold even at increased atmospheric humidity, have a good ability to be washed out and be able to be formulated as optically clear VOC 55 aerosols (i.e. with a VOC fraction of at most 55% by weight). Furthermore, the treated hair should have good haptic properties, such as, for example, a good feel to the touch.

Surprisingly, these objects were achieved by cosmetic preparations comprising at least one polymer A which comprises, in copolymerized form,
a) methyl methacrylate,
b) methacrylic acid,
c) acrylic acid,
d) if appropriate at least one compound with at least two free-radically polymerizable, olefinically unsaturated double bonds, and
e) if appropriate further olefinically unsaturated compounds, with the proviso that the amount by weight of component b) is at least equal to the amount by weight of component c).

Within the scope of the present invention, the expression alkyl comprises straight-chain and branched alkyl groups. Suitable short-chain alkyl groups are, for example, straight-chain or branched C₁-C₁₂-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc. Suitable longer-chain C₈-C₃₀-alkyl or C₈-C₃₀-alkenyl groups are straight-chain and branched alkyl or alkenyl groups. Preferably, these are predominantly linear alkyl radicals, as also occur in natural or synthetic fatty acids and fatty alcohols, and oxo alcohols, which may, if appropriate, be additionally mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

The expression heterocycloalkyl for the purposes of the present invention comprises saturated, cycloaliphatic groups having in general 4 to 7, preferably 5 or 6, ring atoms in which 1 or 2 of the ring carbon atoms are replaced by heteroatoms selected from the elements oxygen, nitrogen and sulfur and which may be optionally substituted, where in the case of a substitution, these heterocycloaliphatic groups can carry 1, 2 or 3, preferably 1 or 2, particularly preferably 1, substituents, selected from alkyl, aryl, COOR, COO⁻M⁺ and NE¹E², preferably alkyl. By way of example of such heterocycloaliphatic groups, mention may be made of pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

Substituted aryl radicals preferably have 1, 2, 3, 4 or 5, in particular 1, 2 or 3, substituents, selected from alkyl, alkoxy, carboxyl, carboxylate, trifluoromethyl, —SO₃H, sulfonate, NE¹E², alkylene-NE¹E², nitro, cyano or halogen.

Hetaryl is preferably pyrrolyl, pyrazolyl, imidazolyl, indolyl, carbazolyl, pyridyl, quinolinyl, acridinyl, pyridazinyl, pyrimidinyl or pyrazinyl.

Arylalkyl is groups which comprise both alkyl and aryl radicals, these arylalkyl groups being linked to the compound carrying them either by the aryl radical or by the alkyl radical.

Preferably, polymer A comprises, based on the total amount of components a) to e), 50-85% by weight, particularly preferably 65-80% by weight and very particularly preferably 70-80% by weight, of methyl methacrylate a) in copolymerized form.

Preferably, polymer A comprises, based on the total amount of components a) to e), preferably 1-30% by weight, particularly preferably 5-25% by weight and very particularly preferably 10-20% by weight, of methacrylic acid b) in copolymerized form. The methacrylic acid can be present either in the form of the free acid or in the form of a cosmetically acceptable salt. Cosmetically acceptable salts are, for example, the alkali metal or ammonium salts of methacrylic acid.

Preferably, polymer A comprises, based on the total amount of components a) to e), preferably 1-20% by weight, particularly preferably 3-15% by weight and very particularly preferably 5-10% by weight, of acrylic acid c) in copolymerized form. The acrylic acid can be present either in the form of the free acid or in the form of a cosmetically acceptable salt. Cosmetically acceptable salts are, for example, the alkali metal or ammonium salts of acrylic acid.

If appropriate, polymer. A comprises, in copolymerized form, at least one further compound d) with at least two free-radically polymerizable, olefinically unsaturated double bonds. Preferably, the amount of this copolymerized component d), based on the total amount of components a) to e), is 0-5, particularly preferably 0-4 and very particularly preferably 0.5-3% by weight.

If appropriate, polymer A comprises further olefinically unsaturated compounds e) in copolymerized form. Preferably, the amount of this at least one copolymerized component d), based on the total amount of components a) to e), is 0-30% by weight, particularly preferably 0-20% by weight and very particularly preferably 0-14.5% by weight.

### Component d)

Components d) are compounds with at least two free-radically polymerizable, olefinically unsaturated double bonds per molecule. Such compounds are usually referred to as crosslinkers.

Free-radically polymerizable, olefinically unsaturated double bonds are, for example, alkenyl groups which arise formally by detaching an H atom from an alkene. These include vinyl (—CH═CH₂), 1-propenyl (—CH═CH—CH₃), 2-propenyl or allyl (—CH₂—CH═CH₂), 1-butenyl (—CH═CH—CH₂—CH₃) etc.

Alkylidene groups, i.e. groups which are joined to a carbon atom of a molecule by a double bond, also belong to the free-radically polymerizable, olefinically unsaturated double bonds (example ethylidene: ═CHCH₃).

Suitable components d) are, for example, acrylic esters, methacrylic esters, ally ethers or vinyl ethers of at least dihydric alcohols. The OH groups of the parent alcohols may here be completely or partially etherified or esterified; however, components d) comprise at least two olefinically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic acid neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000.

Apart from the homopolymers of ethylene oxide and propylene oxide, it is also possible to use block copolymers of ethylene oxide or propylene oxide or copolymers which comprise ethylene oxide and propylene oxide groups in the incorporated form.

Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates. The polyhydric alcohols can also firstly be converted to the corresponding glycidyl ethers by reaction with epichlorohydrin.

Further suitable components d) are the vinyl esters or the esters of monohydric, unsaturated alcohols with ethylenically unsaturated C₃- to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, the monohydric, unsaturated alcohols can also be esterified with polybasic carboxylic acids, for example malonic acid, tartaric acid, trimellitic acid, phthalic acid, terephthalic acid, citric acid or succinic acid.

Further suitable components d) are esters of unsaturated carboxylic acids with the abovedescribed polyhydric alcohols, for example oleic acid, crotonic acid, cinnamic acid or 10-undecenoic acid.

Suitable components d) are furthermore straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Also suitable as components d) are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

Also suitable as components d) are triallylamine and triallylmonoalkylammonium salts, e.g. triallylmethylammonium chloride or methylsulfate.

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Also suitable are alkylenebisacrylamides, such as methylenebisacrylamide and N,N′-(2,2-)-butane and 1,1′-bis(3,3′-vinylbenzimidazolith-2-one)-1,4-butane.

Other suitable components d) are, for example, alkylene glycol di(meth)acrylates, such as ethylene glycol diacrylate, ethylene glycol dimethacrylate, tetraethylene glycol acrylate, tetraethylene glycol dimethacrylate, diethylene glycol acrylate, diethylene glycol methacrylate, vinyl acrylate, allyl acrylate, allyl methacrylate, divinyldioxane, pentaerythritol allyl ether, and mixtures of these components d).

Further suitable components d) are divinyldioxane, tetraallylsilane or tetravinylsilane.

Particularly preferably used components d) are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred components d) are allyl methacrylate, pentaerythritol triallyl ether, methylenebisacrylamide, N,N′-divinylethyleneurea, triallylamine and triallylmonoalkylammonium salts, and acrylic esters of glycol, butanediol, trimethylolpropane or glycerol or acrylic esters of glycol, butanediol, trimethylolpropane or glycerol reacted with ethylene oxide and/or epichlorohydrin.

Mixtures of the abovementioned compounds can of course also be used. Component d) is preferably soluble in the reaction medium. If the solubility of component d) in the reaction medium is low, then it can be dissolved in one monomer or in a monomer mixture or else be metered in dissolved in a solvent which is miscible with the reaction medium. Particular preference is given to those components d) which are soluble in the monomer mixture.

Based on the total amount of components a) to e), components d) are used in amounts of from 0 to 5% by weight, preferably 0 to 4% by weight; very particularly preferably 0.1 to 3% by weight and in particular from 0.5 to 3% by weight.

### Component e)

Suitable as component e) are, in general, all free-radically polymerizable, olefinically unsaturated compounds that are different from components a) to d) and that can be copolymerized with components a) to d).

### Preferred Components e) are

e1) anionic and anionogenic compounds different from b) and c)

e2) esters of (meth)acrylic acid different from a)

e3) compounds containing amide groups different from e4),

e4) (meth)acrylamides,

e5) cationogenic monomers,

e6) cationic monomers.

e1) Anionic and Anionogenic Compounds Different from b) and c)

For the purposes of the present invention, an anionogenic compound is understood as meaning a compound which can be converted to the corresponding anionic form through deprotonation with customary, preferably cosmetically acceptable organic or inorganic bases.

Components e1) are olefinically unsaturated, free-radically polymerizable carboxylic, sulfonic or phosphonic acids and organic and inorganic salts thereof.

Examples of preferred sulfonic acids are 2-acrylamido-2-methylpropanesulfonic acid (AMPS), styrenesulfonic acid, vinylsulfonic acid and salts thereof.

Examples of preferred phosphonic acids are vinylphosphonic acid, 2-acrylamido-2-methylpropanephosphonic acid, allylphosphonic acid and salts thereof.

Component e1) is preferably selected from the group of olefinically unsaturated, free-radically polymerizable carboxylic acids and organic and inorganic salts thereof. The carboxylic acids may be monocarboxylic acids, dicarboxylic acids, carboxylic anhydrides or half-esters of dicarboxylic acids.

Component e1) is particularly preferably selected from the group consisting of ethacrylic acid, alpha-chloroacrylic acid, crotonic acid, maleic acid, maleic anhydride, itaconic acid, citraconic acid, mesaconic acid, glutaconic acid, aconitic acid, fumaric acid, half-esters of olefinically unsaturated dicarboxylic acids having 4 to 10, preferably 4 to 6, carbon atoms and salts thereof.

e2) Esters of (meth)acrylic acid different from a)

Component e2) is selected, for example, from the group consisting of ethyl(meth)acrylate, n-propyl(meth)acrylate, isopropyl(meth)acrylate, n-butyl (meth)acrylate, tert-butyl(meth)acrylate, isobutyl(meth)acrylate, sec-butyl (meth)acrylate, 2-pentyl(meth)acrylate, 3-pentyl(meth)acrylate, isopentyl (meth)acrylate, neopentyl(meth)acrylate, n-octyl(meth)acrylate, 1,1,3,3-tetramethylbutyl(meth)acrylate, ethylhexyl(meth)acrylate, n-nonyl(meth)acrylate, n-decyl (meth)acrylate, n-undecyl(meth)acrylate, tridecyl(meth)acrylate, myristyl (meth)acrylate, pentadecyl(meth)acrylate, palmityl(meth)acrylate, heptadecyl (meth)acrylate, nonadecyl(meth)acrylate, arrachinyl(meth)acrylate, behenyl (meth)acrylate, lignocerenyl(meth)acrylate, cerotinyl(meth)acrylate, melissinyl (meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate, phenoxyethyl (meth)acrylate, 4-t-butylcyclohexyl acrylate, cyclohexyl(meth)acrylate, ureido (meth)acrylate, tetrahydrofurfuryl(meth)acrylate and mixtures thereof.

Component e2) can also be selected from esters of (meth)acrylic acid with alkanediols. These are, for example, 2-hydroxyethyl(meth)acrylate, 2-hydroxyethyl ethacrylate, 2-hydroxypropyl(meth)acrylate, 3-hydroxypropyl(meth)acrylate, 3-hydroxybutyl(meth)acrylate, 4-hydroxybutyl(meth)acrylate, 6-hydroxyhexyl(meth)acrylate, 3-hydroxy-2-ethylhexyl(meth)acrylate, neopentyl glycol mono(meth)acrylate, 1,5-pentanediol mono(meth)acrylate and 1,6-hexanediol mono(meth)acrylate.

Preferred (meth)acrylates are C₂-C₁₀-, particularly preferably C₂-C₈- and in particular C₂-C₄-alkyl (meth)acrylates. Component e) can also be a mixture of methacrylates and acrylates.

e3) Compounds Containing Amide Groups Different from e4)

The compounds e3) containing amide groups are preferably selected from compounds of the general formula VI different from e4)

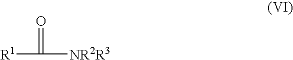

where R¹ is a group of the formula CH₂═CR⁴— where R⁴═H or C₁-C₄-alkyl, and R² and R³, independently of one another, are H, alkyl, cycloalkyl, heterocycloalkyl, aryl or hetaryl, or R² and R³, together with the nitrogen atom to which they are bonded, are a five- to eight-membered nitrogen heterocycle, or

R² is a group of the formula CH₂═CR⁴—, and R¹ and R³, independently of one another, are H, alkyl, cycloalkyl, heterocycloalkyl, aryl or hetaryl, or R¹ and R³, together with the amide group to which they are bonded, are a lactam having 5 to 8 ring atoms.

N-Vinyllactams are preferred as component e3). Suitable components e3) are unsubstituted N-vinyllactams and N-vinyllactam derivatives, which can have for example, one or more C₁-C₆-alkyl substituents, such as methyl, ethyl, n-propyl, isopropyl, n-butyl, sec-butyl, tert-butyl etc. These include, for example, N-vinylpyrrolidone; N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc, and mixtures thereof.

Preferred components e3) are those for which in formula VI R² is CH₂═CH— and R¹ and R³, together with the amide group to which they are bonded, are a lactam having 5 ring atoms.

Particular preference is given to using N-vinylpyrrolidone, N-vinylcaprolactam, N-vinylformamide, acrylamide or mixtures thereof, where N-vinylpyrrolidone is most preferred.

e4) (Meth)acrylamides

Suitable components e4) are the amides of (meth)acrylic add different from e5) and e6). Such amides are, for example, (meth)acrylamide, N-methyl(meth)acrylamide, N-ethyl(meth)acrylamide, N-n-propyl(meth)acrylamide, N-i-propyl(meth)acrylamide, N-(n-butyl)methacrylamide, N-(sec-butyl)methacrylamide, N-(tert-butyl)methacrylamide, N-(n-pentyl)(meth)acrylamide, N-(n-hexyl)(meth)acrylamide, N-(n-heptyl)(meth)acrylamide, N-(n-octyl)(meth)acrylamide, N-(tert-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide.

Suitable components e4) are furthermore 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

e5) and e6) Cationogenic and Cationic Monomers

The components e5) and e6) are monomers which comprise at least one cationogenic and/or cationic group per molecule. A cationogenic group is understood as meaning a structural element which can be converted to the corresponding cationic form by protonation or quaternization.

The cationogenic or cationic groups are preferably nitrogen-containing groups, such as primary, secondary and tertiary amino groups, and quaternary ammonium, groups. The nitrogen-containing groups are preferably tertiary amino groups.

Preference is given to using the components e5) and e6) in uncharged form for the polymerization. However, use in charged form is also suitable.

Charged cationic groups can be produced, for example, from the amine nitrogen atoms by protonation, for example with monobasic or polybasic carboxylic acids, such as lactic acid or tartaric acid, or mineral acids, such as phosphoric acid, sulfuric acid and hydrochloric acid.

Preferably, the components e5) and e6) are selected from
esters of α,β-olefinically unsaturated mono- and dicarboxylic acids with amino alcohols which can be mono- or dialkylated on the amine nitrogen,
amides of α,β-olefinically unsaturated mono- and dicarboxylic acids with diamines which have at least one primary or secondary amino group,
N,N-diallylamine,
N,N-diallyl-N-alkylamines and derivatives thereof,
vinyl- and allyl-substituted nitrogen heterocycles,
vinyl- and allyl-substituted heteroaromatic compounds and
mixtures thereof.

Also suitable as components e5) and e6) are the esters of α,β-olefinically unsaturated mono- and dicarboxylic acids with amino alcohols. Preferred amino alcohols are C₂-C₁₂-amino alcohols which are C₁-C₈-mono- or -dialkylated on the amine nitrogen. Suitable acid components of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof.

Particular preferred components e5) and e6) are N-methylaminoethyl(meth)acrylate, N-ethylaminoethyl(meth)acrylate, N-(n-propyl)aminoethyl(meth)acrylate, N-(n-butyl)aminoethyl (meth)acrylate, N-(tert-butyl)aminoethyl(meth)acrylate, N,N-dimethylaminomethyl (meth)acrylate, N,N-dimethylaminoethyl(meth)acrylate, N,N-diethylaminoethyl (meth)acrylate, N,N-dimethylaminopropyl(meth)acrylate, N,N-diethylaminopropyl (meth)acrylate and N,N-dimethylaminocyclohexyl(meth)acrylate.

In particular, N-(tert-butyl)aminoethyl acrylate and N-(tert-butyl)aminoethyl methacrylate are used as components e5) and e6).

Suitable components e5) and e6) are furthermore the amides of the above-mentioned α,β-olefinically unsaturated mono- and dicarboxylic acids with diamines which have at least one primary or secondary amino group.

Preference is given to diamines which have one tertiary and one primary or secondary amino group. As components e5) and e6), preference is given to using N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide.

Particular preference is given to using N-[3-(dimethylamino)propyl]acrylamide and/or N-[3-(dimethylamino)propyl]methacrylamide.

Suitable components e5) and e6) are furthermore N,N-diallylamines and N,N-diallyl-N-alkylamines and acid addition salts thereof. Alkyl here is preferably C₁-C₂₄-alkyl. Preference is given to N,N-diallyl-N-methylamine.

Suitable components e5) and e6) are furthermore vinyl- and allyl-substituted nitrogen heterocycles, such as N-vinylimidazole, N-vinylimidazole derivatives, for example N-vinyl-2-methylimidazole, vinyl- and allyl-substituted heteroaromatic compounds, such as 2- and 4-vinylpyridine, 2- and 4-allylpyridine, and the salts thereof.

Suitable components e5) and e6) are also N-vinylimidazoles of the general formula VII, in which R¹ to R³ are hydrogen, C₁-C₄-alkyl or phenyl

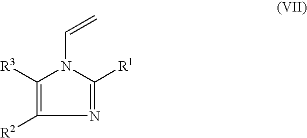

### Examples of Compounds of the General Formula VII can be Found in Table 1 Below:

TABLE 1

| R¹: | R²: | R³: |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl;
Ph = phenyl

The components e5) and e6) are particularly preferably selected from N-(tert-butylamino)ethyl(meth)acrylate, N,N-dimethylaminoethyl(meth)acrylate, N-[3-(dimethylamino)propyl](meth)acrylamide, vinylimidazole and mixtures thereof.

If the polymers A according to the invention comprise components d3) and/or d4) in copolymerized form, then they comprise at least 0.1% by weight, preferably at least 1% by weight, particularly preferably at least 2% by weight and in particular at least 3% by weight and at most 30% by weight, preferably at most 20% by weight, particularly preferably at most 15% by weight and in particular at most 10% by weight, of the components d3) and/or d4), based on the total weight of components a) to d) used.

The charged cationic groups can be produced from the amine nitrogens by quaternization with so-called alkylating agents. Examples of suitable alkylating agents are C₁-C₄-alkyl halides or sulfates, such as ethyl chloride, ethyl bromide, methyl chloride, methyl bromide, dimethyl sulfate and diethyl sulfate. A quaternization can generally take place either before or after the polymerization.

Also suitable as component e) are vinyl acetate, vinyl propionate, vinyl butyrate, ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The components e) can also comprise silicone-containing structural elements.

### Preparation of the Polymers a According to the Invention.

The polymers A according to the invention can be prepared, for example, by solution polymerization, precipitation polymerization, suspension polymerization or emulsion polymerization. Methods of this type are known in principle to the person skilled in the art.

Preference is given to preparation by solution polymerization. It is preferred to prepare the polymers A by free-radical solution polymerization.

Preferred solvents for the polymerization are alcoholic or alcoholic/aqueous solvents such as ethanol and mixtures of ethanol with water and/or further alcohols such as methanol, n-propanol, isopropanol, n-butanol, sec-butanol, tert-butanol, n-hexanol and cyclohexanol, and glycols such as ethylene glycol, propylene glycol and butylene glycol, and the methyl or ethyl ethers of dihydric alcohols such as diethylene glycol, triethylene glycol, polyethylene glycols with number-average molecular weights up to about 3000, glycerol and dioxane.

Particular preference is given to polymerization in alcohol, for example in ethanol or in an alcohol/water mixture, for example in an ethanol/water mixture.

The polymerization temperatures are preferably in a range from about 30 to 140° C., particularly preferably 40 to 120° C. The polymerization usually takes place under atmospheric pressure, although it can also proceed under reduced or increased pressure. A suitable pressure range is between 1 and 5 bar.

### Initiators

For the copolymerization, the monomers can be polymerized with the help of free-radical-forming initiators.

Initiators for the free-radical polymerization which may be used are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, 2,2′-azobis(2-amidinopropane) hydrochloride (Wako V-50®), 2,2′-azobis[2-(2-imidazolin-2-yl)propane] (Wako VA-061®), 2,2′-azobis(2-methylbutyronitrile) (Wako V-59®), dimethyl 2,2′-azobis(2-methylpropionate) (Wako V-601®), 2,2′-azobis(2,4-dimethylvaleronitrile), 1,1′-azobis(1-cyclohexanecarbonitrile), 4,4′-azobis(4-cyanovaleric acid) or 2-(carbamoylazo)-isobutyronitrile.

Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{l}, H₂O₂/ascorbic acid.

Suitable oxidizing agents for redox initiator systems are essentially the abovementioned peroxides. Corresponding reducing agents which can be used are sulfur compounds with a low oxidation state, such as alkali metal sulfites, for example potassium and/or sodium sulfite, alkali metal hydrogen sulfites, for example potassium and/or sodium hydrogen sulfite, alkali metal metabisulfites, for example potassium and/or sodium metabisulfite, formaldehyde sulfoxylates, for example potassium and/or sodium formaldehyde sulfoxylate, alkali metal salts, specifically potassium and/or sodium salts of aliphatic sulfinic acids and alkali metal hydrogen sulfides, such as, for example, potassium and/or sodium hydrogen sulfide, salts of polyvalent metals, such as iron(II) sulfate, iron(II) ammonium sulfate, iron(II) phosphate, enediols, such as dihydroxymaleic acid, benzoin and/or ascorbic acid, and reducing saccharides, such as sorbose, glucose, fructose and/or dihydroxyacetone.

It may also be advantageous to use mixtures of water-soluble initiators and initiators which are sparingly soluble or insoluble in water.

Suitable initiators are described in chapters 20 and 21 of Macromolecules, Vol. 2, 2nd Ed., H. G. Elias, Plenum Press, 1984, New York, to which reference is hereby made in its entirety. Furthermore, suitable photoinitiators are described in S. P. Pappas, J. Rad. Cur., July 1987, p. 6, to which reference is hereby made in its entirety.

The initiators are usually used in amounts up to 10% by weight, preferably 0.02 to 5% by weight, based on the monomers to be polymerized.

### K Value

The K value of the polymers is in the range from 15 to 120, preferably from 25 to 75 and particularly preferably from 25 to 55. K values in the range from 25 to 45 are most preferred (determination in accordance with Fikentscher, Cellulosechemie, Vol. 13, pp. 58 to 64 (1932)).

Ways of adjusting the K value of polymers to a value within a desired range are known to the person skilled in the art. These are, for example, the polymerization temperature, the amount of initiator or the use of chain transfer reagents (regulators).

### Chain Transfer Reagents

To adjust the molecular weight, the polymerization can take place in the presence of at least one chain transfer reagent Chain transfer reagents which can be used are the customary compounds known to the person skilled in the art, such as, for example, sulfur compounds, e.g. mercaptoethanol, 2-ethylhexyl thioglycolate, thioglycolic acid, alkanethiols, cysteine, acetylcysteine, and tribromochloromethane or other compounds which have a regulating effect on the molecular weight of the polymers obtained.

The regulators are usually used in amounts of from 0.1 to 5% by weight, in particular 0.25 to 2% by weight, based on the monomers to be polymerized. Usually, the regulators are added to the polymerization together with the monomers.

### Polymerization Conditions

To achieve the purest possible polymers A with a low residual monomer content, the polymerization (main polymerization) can be followed by at least one afterpolymerization step. The afterpolymerization can take place in the presence of the same initiator system as the main polymerization or a different initiator system. The afterpolymerization preferably takes place at least at the same temperature, preferably at a higher temperature, than the main polymerization. If desired, the reaction mixture can be subjected, following the polymerization or between the first and the second polymerization stages, to stripping with steam or to a steam distillation.

The copolymerization takes place in accordance with the customary processing techniques of solution polymerization, e.g. in accordance with the so-called batch polymerization, which involves initially introducing the monomers and, if appropriate, polymerization regulator and initiator in a solvent, and heating to the polymerization temperature. The reaction mixture is preferably stirred at the polymerization temperature until the conversion of the monomers is more than 99.9%. The initiators can also be added, if appropriate, in these processes only after the polymerization temperature has been reached.

Further process variants are feed methods, which are preferably used. In this case, some or all of the reactants are added, in their entirety or in part, to a reaction mixture batchwise or continuously, together or in separate feeds. Thus, for example, if appropriate, a solution of the polymerization regulator and an initiator solution can be added continuously or batchwise to a mixture of the monomers and a solvent at the polymerization temperature within a given time. However, it is also possible to meter a mixture of initiator and, if appropriate, regulator into the initial charge heated to polymerization temperature. Another variant consists in adding the initiator to the initial charge below or at the polymerization temperature and, if a regulator is to be used, only introducing the regulator or a solution of the regulator into the reaction mixture within a pregiven period after the polymerization temperature has been reached.

The organic solvent used in the production of the polymers can be removed by customary methods known to the person skilled in the art, for example by distillation at reduced pressure. The mixtures that form during the polymerization can be subjected to a physical or chemical aftertreatment following the polymerization process. Such methods are, for example, the known methods for residual monomer reduction, such as, for example, aftertreatment by adding polymerization initiators or mixtures of two or more polymerization initiators at suitable temperatures or heating, the polymerization solution to temperatures above the polymerization temperature, aftertreatment of the polymer solution by means of steam or stripping with nitrogen or treating the reaction mixture with oxidizing or reducing reagents, adsorption methods, such as the adsorption of contamination onto selected media such as, for example, activated carbon, or ultrafiltration. The known work-up steps can also follow, for example suitable drying methods such as spray-drying, freeze-drying or drum-drying, or agglomeration methods that follow the drying. The mixtures with a low content of residual monomer obtained by the method according to the invention can also be put onto the market directly.

Pulverulent polymers have the advantage of better storability, easier transportability and generally exhibit a lower propensity for microbial attack.

### Neutralization

The polymers A according to the invention can be partially or completely neutralized. Particularly for using the polymers in hair cosmetic preparations, partial or complete neutralization is advantageous. In preferred embodiments, the polymers are neutralized for example to at least 10%, preferably to at least 30%, further preferably to at least 40%, particularly preferably to at least 50%, very particularly preferably to at least 70% and in particular to at least 95%.

In a very particularly preferred embodiment, the polymers are neutralized to at least 99%. Most preferably, the neutralization is to at least 100%.

The neutralization can take place during or after the polymerization.

It is also advantageous if the neutralizing agent is added in more than an equivalent amount, equivalent amount being understood as meaning that amount which is at least required in order to neutralize all neutralizable groups of the polymers.

The neutralization can take place, for example, with
a mono-, di- or trialkanolamine having 2 to 5 carbon atoms in the alkanol radical, which is present, if appropriate, in etherified form, for example mono-, di- and triethanolamine, mono-, di- and tri-n-propanolamine, mono-, di-, and triisopropanolamine, 2-amino-2-methylpropanol and di(2-methoxyethyl)amine,
an alkanediolamine having 2 to 5 carbon atoms, for example 2-amino-2-methylpropane-1,3-diol and 2-amino-2-ethylpropane-1,3-diol, or
a primary, secondary or tertiary alkylamine having in total 5 to 10 carbon atoms, for example N,N-diethylpropylamine or 3-diethylamino-1-propylamine.

Suitable alkali metal hydroxides for the neutralization are primarily sodium hydroxide or potassium hydroxide and ammonium hydroxide.

Good neutralization results are often obtained with 2-amino-2-methylpropanol, triisopropanolamine, 2-amino-2-ethylpropane-1,3-diol, N,N-dimethylaminoethanol or 3-diethylamino-1-propylamine.

In a preferred embodiment of the invention, for the neutralization, amines comprising hydroxy groups are selected from the group consisting of N,N-dimethylethanolamine, N-methyldiethanolamine, triethanolamine, 2-amino-2-methylpropanol and mixtures thereof.

In this connection, secondary or tertiary amino-group-carrying alkanolamines can exhibit advantageous effects.

For the neutralization of the polymers in the preparations and compositions according to the invention, silicone polymers comprising amino groups in particular are suitable. Suitable silicone polymers comprising amino groups are, for example, the silicone-aminopolyalkylene oxide block copolymers of WO 97/32917, the products Silsoft®A-843 (dimethicone bisamino hydroxypropyl copolyol) and Silsoft®A-858 (trimethylsilyl amodimethicone copolymer) (both Witco). Of further suitability are also the neutralization polymers of EP-A 1 035 144 and, in particular, the silicone-containing neutralization polymers according to claim 12 of EP-A 1 035 144.

To increase the storage stability of the polymer solutions, it is advantageous to partially neutralize the polymers following preparation. A neutralization immediately after the polymerization in the range from 10 to 20 mol %, based on the total amount of acid groups, is particularly advantageous.

### Cosmetic Preparations

The term VOC is known to the person skilled in the art. VOC (volatile organic compounds) are organic-chemical compounds which boil under atmospheric pressure in a range up to about 260° C. and can thus pass into the ambient air in gaseous form. Volatile organic compounds include numerous solvents and propellants.

Classification of organic compounds in an interior space
(according to: WHO—World Health Organization 1989)

| | | Boiling point range |
|---|---|---|
| Compounds | Abbreviation | [° Celsius] |
| very volatile organic compounds | VVOC | <0 to 50° C. |
| volatile organic compounds | VOC | 50 to 260° C. |
| semi volatile organic compounds | SVOC | 260 to 380° C. |

The described polymers A are exceptionally suitable for producing cosmetic, in particular skin cosmetic and/or hair cosmetic, preparations. They serve here, for example, as polymeric film formers. They can be formulated into and used universally in highly diverse cosmetic, preferably hair cosmetic, preparations and are compatible with the customary components.

The polymers A are advantageously suitable for producing elastic hair styles with simultaneously strong hold (even at high atmospheric humidity). The polymers A according to the invention are characterized by good propellant gas compatibility, good solubility in aqueous/alcoholic solvent mixtures, in particular by suitability for use as optically clear low-VOC formulations and by good ability to be washed out and ability to be combed out without flaking effect. Furthermore, they improve hair treated with them in its sensorily perceivable properties, such as feel, volume, handleability etc. Hair spray formulations based on the polymers A according to the invention are characterized by good sprayability and good rheological properties and extremely low stickiness of the resulting films. The cosmetic, preferably hair cosmetic, preparations according to the invention comprising polymer A do not have tendency toward foam formation following application. Besides the good compatibility with the customary cosmetic ingredients, the applied polymers A dry rapidly.

### Cosmetically Acceptable Carrier B)

The cosmetic preparations according to the invention are preferably aqueous preparations which comprise at least 10% by weight, preferably at least 20% by weight and particularly preferably at least 30% by weight, of water. Preferably, the cosmetic preparations according to the invention comprise at most 80% by weight, preferably at most 55% by weight, of volatile organic constituents. Besides water and the polymers A, the cosmetic preparations, according to the invention further have at least one cosmetically acceptable carrier B) which is selected from
i) water-miscible organic solvents, preferably C₂-C₄-alkanols, in particular ethanol,
ii) oils, fats, waxes,
iii) esters of C₆-C₃₀-monocarboxylic acids with mono-, di- or trihydric alcohols different from ii),
iv) saturated acyclic and cyclic hydrocarbons,
v) fatty acids,
vi) fatty alcohols,
vii) propellants (propellant gases) and
viii) mixtures thereof.

Suitable carriers B and further active ingredients and additives to be used advantageously are described in detail below.

Suitable cosmetically and pharmaceutically compatible oil and fat components B) are described in Karl-Heinz Schrader, Grundlagen and Rezepturen der Kosmetika [Fundamentals and formulations of cosmetics], 2nd edition, Verlag Hüthig, Heidelberg, pp. 319-355, to which reference is hereby made. The preparations according to the invention can, for example, have an oil or fat component B) which is selected from: hydrocarbons of low polarity, such as mineral oils; linear saturated hydrocarbons, preferably having more than 8 carbon atoms, such as tetradecane, hexadecane, octadecane etc; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc and mixtures thereof.

Suitable silicone oils. B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are commercially available, for example, under the name Cyclomethicon.

Preferred oil or fat components. B) are selected from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oils, cod-liver oil, pig grease, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic add and saturated, unsaturated and substituted fatty acids different therefrom; waxes, such as beeswax, carnauba wax, candililla wax, spermaceti, and mixtures of the abovementioned oil and fat components.

Suitable hydrophilic carriers B) are selected from water, 1-, 2- or polyhydric alcohols having preferably 1 to 8 carbon atoms, such as ethanol, n-propanol, isopropanol, propylene glycol, glycerol, sorbitol, etc.

The cosmetic preparations according to the invention may be skin cosmetic, hair cosmetic or dermatological, hygiene or pharmaceutical preparations. On account of their film-forming and flexible properties, the polymers A described above are, particularly suitable as additives for hair and skin cosmetics.

Preferably, the preparations according to the invention which comprise the polymers A are in the form of a spray, gel, foam, ointment, cream, emulsion, suspension, lotion, milk or paste. If desired, liposomes or microspheres may also be used.

Preferably, the cosmetic compositions according to the invention comprise at least one polymer A as defined above, at least one carrier B as defined above and at least one constituent different therefrom which is preferably selected from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tints, tanning agents, dyes, pigments, consistency regulators, humectants, refatting agents, collagen, protein hydrolyzates, lipids, antioxidants, antifoams, antistats, emollients and softeners.

The preparations according to the invention preferably have a pH from 2.0 to 9.3. The pH range is particularly preferably between 4 and 8. Additional cosolvents which may be present are organic solvents or a mixture of solvents with a boiling point below 400° C. in an amount of from 0.1 to 15% by weight, preferably from 1 to 10% by weight. Particularly suitable additional cosolvents are unbranched or branched hydrocarbons, such as pentane, hexane, isopentane and cyclic hydrocarbons, such as cyclopentane and cyclohexane. Further particularly preferred water-soluble solvents are glycerol, ethylene glycol and propylene glycol in an amount up to 30% by weight.

In a preferred embodiment of the invention, the preparations according to the invention have a fraction of volatile organic components of at most 80% by weight, preferably at most 55% by weight and in particular at most 35% by weight. A preferred subject matter are thus cosmetic, preferably hair cosmetic, preparations which correspond to the low-VOC standard, i.e. VOC-80 or VOC-55 standard.

Preference is given to the use of the polymers A in particular in hair spray preparations which comprise the following constituents:
partially or completely neutralized polymer A according to the invention;
water;
cosmetically customary organic solvent, such as, for example, ethanol, isopropanol and dimethoxymethane, in addition also acetone, n-propanol, n-butanol, 2-methoxypropan-1-ol, n-pentane, n-hexane, cyclohexane, n-heptane, n-octane or dichloromethane or mixtures thereof;
cosmetically customary propellants, such as, for example, n-propane, iso-propane, n-butane, isobutane, 2,2-dimethylbutane, n-pentane, isopentane, dimethyl ether, difluoroethane, fluorotrichloromethane, dichlorodifluoromethane or dichlorotetrafluoroethane, HFC-152 A (1,1-difluoroethane), HFC-134a (1,1,2,2-tetrafluoroethane), N₂, N₂O and CO or mixtures thereof.

For the neutralization of the polymers A according to the invention and for adjusting the pH of the cosmetic, preferably hair cosmetic, preparations, alkanolamines are advantageously used. Examples (INCI) are Aminomethylpropanol, Diethanolamine, Diisopropanolamine, Ethanolamine, Methylethanolamine, N-lauryl Diethanolamine, Triethanolamine, Triisopropanolamine, etc. It is possible to use alkanolamines carrying either primary amino groups or secondary amino groups.

Furthermore, alkali metal hydroxides (e.g. NaOH, preferably KOH) and other bases can be used for the neutralization (e.g. histidine, arginine, lysine or ethylenediamines, diethylenetriamine, melamine, benzoguanamine). All of the stated bases can be used on their own or as a mixture with other bases for the neutralization of acid-containing cosmetic products.

In a preferred embodiment of the invention, for the neutralization, amines comprising hydroxy groups are selected from the group consisting of N,N-dimethylethanolamine, N-methyldiethanolamine, triethanolamine, 2-amino-2-methylpropanol and mixtures thereof.

In this connection, alkanolamines carrying secondary or tertiary amino groups can exhibit advantageous effects.

Accordingly, the present invention provides aqueous cosmetic, preferably skin cosmetic and/or hair cosmetic preparations which, besides the at least one polymer A and the carrier B, also comprise at least one active ingredient or additive selected from the group consisting of viscosity-modifying substances, hair care substances, hair-setting substances, silicone compounds, photoprotective substances, fats, oils, waxes, preservatives, pigments, soluble dyes, particulate substances, and surfactants.

In a preferred embodiment, hair cosmetic formulations according to the invention comprise
i) 0.05 to 20% by weight of at least one polymer A,
ii) 20 to 99.95% by weight of water and/or alcohol,
iii) 0 to 50% by weight of at least one propellant gas,
iv) 0 to 5% by weight of at least one emulsifier,
v) 0 to 3% by weight of at least one thickener, and
vi) up to 25% by weight of further constituents.

Alcohol is to be understood as meaning all of the abovementioned alcohols customary in cosmetics, e.g. ethanol, isopropanol, n-propanol.

### Propellants (Propellant Gases)

As propellants (propellant gases), of the specified compounds, especially the hydrocarbons, in particular propane, n-butane, n-pentane and mixtures thereof, and dimethyl ether and difluoroethane are used. If appropriate, one or more of the specified chlorinated hydrocarbons are co-used in propellant mixtures, but only in small amounts, for example up to 20% by weight, based on the propellant mixture.

The cosmetic, preferably hair cosmetic, preparations according to the invention are also particularly suitable for pump spray preparations without the addition of propellants and also for aerosol sprays with customary compressed gases such as nitrogen, compressed air or carbon dioxide as propellant.

A water-containing standard aerosol spray formulation comprises, for example, the following constituents:
polymer A neutralized to 100%
alcohol
water
dimethyl ether and/or propane/n-butane and/or propane/isobutane.

Here, the total amount of the volatile organic components is preferably at most 80% by weight, particularly preferably at most 55% by weight, of the preparation.

Preferably, the cosmetic, preferably hair cosmetic, preparations according to the invention comprise at least one polymer A according to the invention, at least one cosmetically acceptable carrier B) as defined above and at least one further active ingredient or additive different therefrom which is selected from cosmetically active ingredients, emulsifiers, surfactants, preservatives, perfume oils, thickeners, hair polymers, hair conditioners, graft polymers, water-soluble or dispersible silicone-containing polymers, photoprotective agents, bleaches, gel formers, care agents, colorants, tints, tanning agents, dyes, pigments, consistency regulators, humectants, refatting agents, collagen, protein hydrolyzates, lipids, antioxidants, antifoams, antistats, emollients, lanolin components, protein hydrolyzates and softeners.

### Further Polymers

For the targeted adjustment of the properties of cosmetic, preferably hair cosmetic, preparations, it is often advantageous to use the polymers according to the invention in a mixture with further (hair) cosmetically customary polymers.

In a further preferred embodiment, the composition according to the invention comprises 0.01 to 15% by weight, preferably 0.5 to 10% by weight, of at least one further synthetic or natural nonionic, preferably a film-forming, polymer. Natural polymers are also understood as meaning chemically modified polymers of natural origin. Film-forming polymers are understood as meaning those polymers which, when applied in 0.01 to 5% strength aqueous, alcoholic or aqueous-alcoholic solution, are able, to deposit a polymer film on the hair.

Suitable as such further customary polymers for this purpose are, for example, anionic, cationic, amphoteric, zwitterionic and neutral polymers.

Examples of such further polymers are
copolymers of ethyl acrylate and methacrylic acid
copolymers of N-tert-butylacrylamide, ethyl acrylate and acrylic acid
polyvinylpyrrolidones
polyvinylcaprolactams
polyurethanes
copolymers of acrylic acid, methyl methacrylate, octylacrylamide, butylaminoethyl methylacrylate and hydroxypropyl methacrylate,
copolymers of vinyl acetate and crotonic acid and/or (vinyl)neodecanoate,
copolymers of vinyl acetate and/or vinyl propionate and N-vinylpyrrolidone,
carboxyfunctional copolymers of vinylpyrrolidone, t-butyl acrylate, methacrylic acid,
copolymers of tert-butyl acrylate, methacrylic acid and dimethicone copolyol.

Surprisingly, it has been found that cosmetic and preferably hair cosmetic preparations which comprise the polymers A in combination with further polymers have unexpected properties. The cosmetic and preferably hair cosmetic preparations according to the invention are superior to preparations from the prior art especially with regard to their cosmetic properties as a whole.

Copolymers of ethyl acrylate and methacrylic acid (INCI name: Acrylates Copolymer), are available, for example, as commercial products Luviflex® Soft (BASF).

Copolymers of N-tert-butylacrylamide, ethyl acrylate and acrylic acid (INCI name: Acrylates/Acrylamide Copolymer) are available, for example, as commercial products Ultrahold Strong®, Ultrahold 8® (BASF).

Polyvinylpyrrolidones (INCI name: PVP) are available, for example, under the trade names Luviskol®K, Luviskol®K30 (BASF) and PVP K® (ISP).

Polyvinylcaprolactams (INCI: Polyvinylcaprolactams) are available, for example, under the trade name Luviskol Plus® (BASF).

Polyurethanes (INCI: Polyurethane -1) are available, for example, under the trade name Luviset®PUR.

Copolymers of acrylic acid, methyl methacrylate, octylacrylamide, butylaminoethyl methylacrylate, hydroxypropyl methacrylate (INCI: Octylacrylamide/Acrylates/Butylaminoethyl Methacrylate Copolymer) are known, for example, under the trade names Amphomer®28-4910 and Amphomer®LV-71 (National Starch).

Copolymers of vinyl acetate and crotonic acid (INCI: VA/Crotonate/Copolymer) are available, for example, under the trade names Luviset®CA 66 (BASF), Resyn®28-1310 (National Starch), Gafset® (GAF) or Aristoflex®A (Celanese).

Copolymers of vinyl acetate, crotonic acid and (vinyl)neodecanoate (INCI: VA/Crotonates/Neodecanoate Copolymer) are available, for example, under the trade names Resyn®28-2930 (National Starch) and Luviset®CAN (BASF).

Copolymers of vinyl acetate and N-vinylpyrrolidone (INCI: PVPNA) are obtainable, for example, under the trade names Luviskol VA® (BASF) and PVP/VA (ISP).

Carboxyfunctional copolymers of vinylpyrrolidone, t-butyl acrylate, methacrylic acid are available, for example, under the trade name Luviskol®VBM (BASF).

Copolymers of tert-butyl acrylate, methacrylic acid and dimethicone copolyol are available, for example, under the trade name Luviflex®Silk (BASF).

Suitable anionic polymers are homopolymers and copolymers, different from the polymers A, of acrylic acid and methacrylic acid or salts thereof, copolymers of acrylic acid and acrylamide and salts thereof, sodium salts of polyhydroxycarboxylic adds, copolymers of acrylic acid and methacrylic acid with, for example, hydrophobic monomers, e.g. C₄-C₃₀-alkyl esters of (meth)acrylic acid, C₄-C₃₀-alkylvinyl esters, C₄-C₃₀-alkyl vinyl ethers and hyaluronic acid, and further polymers known under the trade names Amerhold®DR-25, Ultrahold®, Luviset®P.U.R., Acronal®, Acudyne®, Lovocryl®, Versatyl®, Amphomer® (28-4910, LV-71), Placise®L53, Gantrez®ES 425, Advantage Plus®, Omnirez®2000, Resyn®28-1310, Resyn®28-2930, Balance® (0/55), Acudyne®255, Aristoflex®A or Eastman AQ®.

Furthermore, the group of suitable polymers comprises, by way of example, Balance®CR (National Starch), Balance®47 (National Starch; octylacrylamide/acrylates/butylaminoethyl methacrylate copolymer), Aquaflex®FX 64 (ISP; isobutylene/ethylmaleimide/hydroxyethylmaleimide copolymer), Aquaflex®SF-40 (ISP/National Starch; VP/vinyl caprolactam/DMAPA acrylate copolymer), Allianz®LT-120 (ISP/Rohm & Haas; acrylate/C1-2 succinate/hydroxyacrylate copolymer), Aquarez® HS (Eastman; Polyester-1).

Also suitable are the polymers under the trade names Diaformer®Z-400 (Clariant; methacryloylethylbetaine/methacrylate copolymer), Diaformer®Z-711 (Clariant; methacryloylethyl N-oxide/methacrylate copolymer), Diaformer®Z-712 (Clariant; methacryloylethyl N-oxide/methacrylate copolymer), Omnirez®2000 (ISP; monoethyl ester of poly(methyl vinyl ether/maleic acid in ethanol), Amphomer®HC (National Starch; acrylate/octylacrylamide copolymer), Amphomer®28-4910 (National Starch; octyl acrylamide/acrylate/butylaminoethyl methacrylate copolymer), Advantage®HC 37 (ISP; terpolymer of vinylcaprolactam/vinylpyrrolidone/dimethylaminoethyl methacrylate), Advantage®LC55 and LC80 or LC A and LC E, Advantage®Plus (ISP; VA/butyl maleate/isobornyl acrylate copolymer), Aculyne®258 (Rohm & Haas; acrylate/hydroxyester acrylate copolymer), Luviset®P.U.R. (BASF, Polyurethane-1), Eastman®AQ 48 (Eastman), Styleze®CC-10 (ISP; VP/DMAPA acrylates copolymer), Styleze® 2000 (ISP; VP/acrylates/lauryl methacrylate copolymer), DynamX® (National Starch; Polyurethane-14 AMP-acrylates copolymer), Resyn®XP (National Starch; acrylates/octylacrylamide copolymer), Fixomer® A-30 (Ondeo Nalco; polymethacrylic acid (and) acrylamidomethylpropanesulfonic acid), Fixate® G-100 (Noveon; AMP-acrylates/allyl methacrylate copolymer).

Suitable polymers are also copolymers of (meth)acrylic acid and polyether acrylates, where the polyether chain is terminated with a C₈-C₃₀-alkyl radical. These include, for example, acrylate/beheneth-25 methacrylate copolymers which are available under the name Aculyn® (Rohm+Haas). Particularly suitable polymers are also copolymers of t-butyl acrylate, ethyl acrylate, methacrylic acid (e.g. Luvimer®100P, Luvimer®Pro55) and copolymers of ethyl acrylate and methacrylic acid (e.g. Luvimer®MAE).

Also suitable are crosslinked polymers of acrylic acid, as are obtainable under the INCI name Carbomer. Such crosslinked homopolymers of acrylic acid are commercially available, for example, as Carbopol® (Noveon). Preference is also given to hydrophobically modified crosslinked polyacrylate polymers, such as Carbopol®Ultrez 21 (Noveon). Such further polymers can also be used for modifying the rheology of the preparations, i.e. as thickeners.

Furthermore suitable as additional polymers are water-soluble or water-dispersible polyesters, polyureas, polyurethanes, polyurethaneureas, maleic anhydride copolymers optionally reacted with alcohols, or anionic polysiloxanes.

Polymers furthermore suitable for use together with the polymers A are, for example, also cationic and cationogenic polymers. These include, for example,
copolymers of N-vinylpyrrolidone/N-vinylimidazolium salts (available, for example, under the trade names Luviquat®FC, Luviquat®HM, Luviquat®MS, Luviquat®Care, Luviquat® UltraCare (BASF),
copolymers of N-vinylcaprolactam/N-vinylpyrrolidone/N-vinylimidazolium salts (available, for example, under the trade name Luviquat®Hold),
copolymers of N-vinylpyrrolidone/dimethylaminoethyl methacrylate, quaternized with diethyl sulfate (available, for example, under the trade name Luviquat®PQ11),
copolymers of vinylpyrrolidone, methacrylamide, vinylimidazole (Luviset®Clear)
cationic cellulose derivatives (Polyquatemium-4 and -10),
acrylamide copolymers (Polyquaternium-7),
guar hydroxypropyltrimethylammonium chloride (INCI: Hydroxypropyl Guar Hydroxypropyltrimonium Chloride),
polyethyleneimines and salts thereof,
polyvinylamines and salts thereof,
polymers based on dimethyldiallylammonium chloride (Merquat®),
polymers which are formed by reacting polyvinylpyrrolidone with quaternary ammonium compounds. (Gafquat®),
hydroxyethylcellulose with cationic groups (Polymer®JR) and
vegetable-based cationic polymers, e.g. guar polymers, such as the Jaguar® grades from Rhodia.

Suitable further hair cosmetic polymers are also neutral polymers such as
polyvinylpyrrolidones,
copolymers of N-vinylpyrrolidone and vinyl acetate and/or vinyl propionate,
polysiloxanes,
polyvinylcaprolactams and
copolymers with N-vinylpyrrolidone,
cellulose derivatives,
polyaspartic acid salts and derivatives,
polyamides, e.g. based on itaconic acid and aliphatic diamines, as described in DE-A-43 33 238:

The abovementioned types of polymer include those known under the trade names Luviskol® (K, VA, Plus), PVP K, PVP/VA, Advantage®HC, Luviflex®Swing, Kollicoat®IR, H₂OLD®EP-1.

Furthermore, suitable as further polymers are also biopolymers, i.e. polymers which are obtained from naturally renewable raw materials and are composed of natural monomer building blocks, e.g. cellulose derivatives and chitin, chitosan, DNA, hyaluronic acid and RNA derivatives.

Suitable mixing partners for the polymers according to the invention are also zwitterionic polymers, as are disclosed, for example, in the German patent applications DE 39 29 973, DE 21 50 557, DE 28 17 369 and DE 37 08 451, and methacroylethylbetaine/methacrylate copolymers which are commercially available under the name Amersette® (Amerchol), or copolymers of hydroxyethyl methacrylate, methyl methacrylate, N,N-dimethylaminoethyl methacrylate and acrylic acid (Jordapon®).

Further suitable polymers are also betainic polymers, such as Yukaformers (R205, SM) and Diaformers.

Polymers suitable as mixing partners are also nonionic, siloxane-containing, water-soluble or -dispersible: polymers, e.g. polyether siloxanes, such as Tegopren® (Goldschmidt) or Belsil® (Wacker).

Cosmetically and/or Dermatologically Active Ingredients

Suitable cosmetically and/or dermatologically active ingredients are, for example, coloring, active ingredients, skin and hair pigmentation agents, tinting agents, tanning agents, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, antioxidative or free-radical-scavenging active ingredients, skin-moisturizing or -humectant substances, refatting active ingredients, antierythematous or antiallergic active ingredients and mixtures thereof.

Preferred cosmetic care and active ingredients are AHA acids, fruit acids, ceramides, phytantriol, collagen, vitamins and provitamins, for example vitamin A, E and C, retinol, bisabolol and panthenol. A particularly preferred cosmetic care substance in the preparations according to the invention is panthenol, which is commercially available, for example, as D-Panthenol®USP, D-Panthenol®50 P, D-Panthenol®75 W, D,L-Panthenol®50 W.

Artificially skin-tanning active ingredients which are suitable for tanning the skin without natural or artificial irradiation with UV rays are, for example, dihydroxyacetone, alloxan and walnut shell extract.

Suitable keratin-hardening substances are generally active ingredients as are also used in antiperspirants, such as, for example, potassium aluminum sulfate, aluminum hydroxychloride, aluminum lactate, etc.

Antimicrobial active ingredients are used in order to destroy microorganisms onto suppress their growth and thus serve both as preservative and deodorizing substance which reduces the formation or the intensity of body odor. These include, for example, customary preservatives known to the person skilled in the art, such as p-hydroxybenzoic acid esters, imidazolidinylurea, formaldehyde, sorbic acid, benzoic acid, salicylic acid, etc. Such deodorizing substances are, for example, zinc ricinoleate, triclosan, undecylenic acid alkylolamides, citric acid triethyl ester, chlorhexidine etc. The preparations according to the invention comprise preferably 0.01 to 5% by weight, particularly preferably 0.05 to 1% by weight, of at least one preservative. Suitable further preservatives are the substances listed in the International Cosmetic Ingredient Dictionary and Handbook, 9th edition with the function “Preservatives”, e.g. phenoxyethanol, benzyl paraben, butyl paraben, ethyl paraben, isobutyl, paraben, isopropyl paraben, methyl paraben, propyl paraben, iodopropynyl butylcarbamate, methyldibromoglutaronitrile, DMDM hydantoin.

### UV Filter Substances

In one embodiment, the preparations according to the invention can comprise oil-soluble and/or water-soluble UVA and/or UVB filters.

The total amount of the filter substances is preferably 0.01 to 10% by weight or from 0.1 to 5% by weight, particularly preferably from 0.2 to 2% by weight, based on the total weight of the preparations.

The majority of the photoprotective agents in the preparations serving to protect the human epidermis consists of compounds which absorb UV light in the UV-B region. For example, the fraction of UV-A absorbers to be used according to the invention is 10 to 90% by weight, preferably 20 to 50% by weight, based on the total amount of UV-B and UV-A absorbing substances.

The UVB filters may be oil-soluble or water-soluble. Advantageous UVB filter substances are, for example:
i) benzimidazolesulfonic acid derivatives, such as, for example, 2-phenylbenzimidazole-5-sulfonic acid and salts thereof;
ii) benzotriazole derivatives, such as, for example, 2,2′-methylenebis(6-(2H-benzotriazol-2-yl)-4-(1,1,3,3-tetramethylbutyl)phenol);
iii) 4-aminobenzoic acid derivatives, preferably 2-ethylhexyl 4-(dimethylamino)-benzoate, amyl 4-(dimethylamino)benzoate;
iv) esters of benzalmalonic acid, preferably di(2-ethylhexyl) 4-methoxybenzalmalonate;
v) esters of cinnamic acid, preferably 2-ethylhexyl 4-methoxycinnamate, isopentyl 4-methoxycinnamate;
vi) derivatives of benzophenone, preferably 2-hydroxy-4-methoxybenzophenone, 2-hydroxy-4-methoxy-4′-methylbenzophenone, 2,2′-dihydroxy-4-methoxybenzophenone;
vii) methylidenecamphor derivatives, preferably 4-methylbenzylidenecamphor, benzylidenecamphor;
viii) triazine derivatives, preferably tris(2-ethylhexyl) 4,4′,4″-(1,3,5-triazine-2,4,6-triylimino)trisbenzoate [INCI: Diethylhexyl Butamido Triazine, UVA-Sorb® HEB (Sigma 3V)] and 2,4,6-tris[anilino-(p-carbo-2′-ethyl-1-hexyloxy)]-1,3,5-triazine [INCI: Octyl Triazone, Uvinul® 150 (BASF)].

Water-soluble UVB filter substances to be used advantageously are, for example, sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

UVA filters to be used advantageously are, for example:
1,4-phenylenedimethinecamphorsulfonic acid derivatives, such as, for example, 3,3′-(1,4-phenylenedimethine)bis(7,7-dimethyl-2-oxobicyclo[2.2.1]heptane-1-methanesulfonic acid and its salts
1,3,5-triazine derivatives, such as 2,4-bis{[(2-ethylhexyloxy)-2-hydroxy)phenyl}-6-(4-methoxyphenyl)-1,3,5)triazine (e.g. Tinosorb®S (Ciba))
dibenzoylmethane derivatives, preferably 4-isopropyldibenzoylmethane, 4-(tert-butyl)-4′-methoxydibenzoylmethane
benzoxazole derivatives, for example 2,4-bis[445-(1,1-dimethylpropyl)benzoxazol-2-yl]phenylimino]-6-[(2-ethylhexyl)imino]-1,3,5-triazine (CAS No. 288254-1 6-0, Uvasorb®K2A (3V Sigma))
hydroxybenzophenones, for example hexyl 2-(4′-diethylamino-2′-hydroxybenzoyl)-benzoate (also: Aminobenzophenone) (Uvinul®A Plus (BASF))

Furthermore, according to the invention, it may, if appropriate, be advantageous to provide preparations with further UVA and/or UVB filters, for example certain salicylic acid derivatives, such as 4-isopropylbenzyl salicylate, 2-ethylhexyl salicylate, octyl salicylate, homomethyl salicylate. The total amount of salicylic acid derivatives in, the cosmetic preparations is advantageously selected from the range from 0.1-15.0% by weight, preferably 0.3-10.0% by weight, based on the total weight of the preparations. A further photoprotective filter to be used advantageously according to the invention is ethylhexyl 2-cyano-3,3-diphenylacrylate (Octocrylen, Uvinul®N 539 (BASF)).

The table below summarizes by way of example a number of photoprotective filters suitable for use in the preparations according to the invention:

| No. | Substance | CAS No. |
|---|---|---|
| 1 | 4-Aminobenzoic acid | 150-13-0 |
| 2 | 3-(4′-Trimethylammonium)benzylidenebornan-2-one | 52793-97-2 |
| | methylsulfate | |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its | |
| | salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-ethylhexyl 2-cyano-3,3-diphenylacrylate | 6197-30-4 |
| 20 | Menthyl o-aminobenzoate or: | 134-09-8 |
| | 5-methyl-2-(1-methylethyl)-2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (Dioxybenzone) | 131-53-3 |
| 23 | 2-Hydroxy-4-methoxy-4-methylbenzophenone | 1641-17-4 |
| | (Mexenone) | |
| 24 | Triethanolamine salicylate | 2174-16-5 |
| 25 | Dimethoxyphenylglyoxalic acid or: | 4732-70-1 |
| | 3,4-dimethoxyphenylglyoxalacidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3- | 103597-45-1 |
| | tetramethylbutyl)phenol] | |
| 30 | 2,2′-(1,4-Phenylene)bis-1H-benzimidazole-4,6- | 180898-37-7 |
| | disulfonic acid, Na salt | |
| 31 | 2,4-bis-[4-(2-ethylhexyloxy)-2-hydroxy]phenyl- | 187393-00-6 |
| | 6-(4-methoxyphenyl)-(1,3,5)-triazine | |
| 32 | 3-(4-methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone-5,5′-disodium | 3121-60-6 |
| | sulfonate | |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3- | 155633-54-8 |
| | tetramethyl-1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Suitable UV photoprotective filters with the CAS No. 113010-52-9 are commercially available, for example, under the name Uvinul®P 25.

Polymeric or polymer-bonded filter substances can also be used according to the invention.

Metal oxides, such as titanium dioxide or zinc oxide, can likewise be used advantageously to protect against harmful solar radiation. Their effect is based essentially on reflection, scattering and absorption of the harmful UV radiation and essentially depends on the primary particle size of the metal oxides. The cosmetic preparations according to the invention can furthermore advantageously comprise inorganic pigments based on metal oxides and/or other metal compounds that are insoluble or sparingly soluble in water, selected from the group of oxides of zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. They are particularly preferably pigments based on ZnO.

The inorganic pigments here may be present in coated form, i.e. they are surface-treated. This surface treatment can consist, for example, in providing the pigments with a thin hydrophobic layer by a method known per se, as described in DE-A-33 14 742. Photoprotective agents suitable for use in the preparations according to the invention are the compounds specified in EP-A 1 084 696 in paragraphs [0036] to [0053], to which reference is made at this point in its entirety. Of suitability for the use according to the invention are all UV photoprotective filters which are specified in Annex 7 (to §3b) of the German Cosmetics Ordinance under “Ultraviolet filters for cosmetic compositions”.

The list of specified UV photoprotective filters which can be used in the preparations according to the invention is not exhaustive.

### Thickeners

Suitable thickeners are specified in “Kosmetik and Hygiene von Kopf bis Full” [Cosmetics and Hygiene from head to toe”], ed. W. Umbach, 3rd edition, Wiley-VCH, 2004, pp. 235-236, to which reference is made at this point in its entirety.

Consistency regulators permit the adjustment of the desired viscosity of, for example, shampoos. Thickeners, which have a viscosity-building effect as a result of increasing size of the surfactant micelles or as a result of swelling the water phase, originate from chemically very different classes of substances.

Suitable thickeners for the preparations according to the invention are, for example, crosslinked polyacrylic acids and derivatives thereof, polysaccharides, such as xanthan gum, guar guar, agar agar, alginates or tyloses, cellulose derivatives, for example carboxymethylcellulose or hydroxycarboxymethylcellulose, also relatively high molecular weight polyethylene glycol mono- and diesters of fatty acids, fatty alcohols, monoglycerides and fatty acids, polyvinyl alcohol and polyvinylpyrrolidone.

Suitable thickeners are commercially available under the tradenames Carbopol®(Noveon), Ultrez® (Noveon), Luvigel® EM (BASF), Capigel®98 (Seppic), Synthalene® (Sigma), Aculyn® (Rohm and Haas) such as Aculyn® 22 (copolymer of acrylates and methacrylic acid ethoxylates with stearyl radical (20 EO units)) and Aculyn® 28 (copolymer of acrylates and methacrylic acid ethoxylates with behenyl radical (25 EO units)).

Suitable thickeners are furthermore, for example, Aerosil grades (hydrophilic silicas), polyacrylamides, polyvinyl alcohol and polyvinylpyrrolidone, surfactants, such as, for example, ethoxylated fatty acid glycerides, esters of fatty acids with polyols, such as, for example, pentaerythritol or trimethylolpropane, fatty alcohol ethoxylates with a narrowed homolog distribution or alkyl oligoglucosides, and electrolytes, such as sodium chloride and ammonium chloride.

Particularly preferred thickeners for producing gels are Ultrez®21, Aculyn®28, Luvigel® EM and Capigel®98.

Particularly in the case of more highly concentrated shampoo formulations, to regulate the consistency, it is also possible to add substances which reduce the viscosity of the formulation, such as, for example, propylene glycol or glycerol. These substances only slightly influence the product properties.

### Gel Formers

If the use of gel formers is desired for the preparations according to the invention, then it is possible to use all gel formers customary in cosmetics. These include lightly crosslinked polyacrylic acid, for example carbomer (INCI), cellulose derivatives, e.g. hydroxypropylcellulose, hydroxyethylcellulose, cationically modified celluloses, polysaccharides, e.g. xanthum gum, caprylic/capric triglycerides, sodium acrylates copolymer, Polyquaternium-32 (and) Paraffinum Liquidum (INCI), sodium acrylates copolymer (and) Paraffinum Liquidum (and) PPG-1 trideceth-6, acrylamidopropyl trimonium chloride/acrylamide copolymer, steareth-10 allyl ether acrylates copolymer, Polyquaternium-37 (and) Paraffinum Liquidum (and) PPG-1 Trideceth-6, Polyquaternium 37 (and) Propylene Glycol Dicaprate Dicaprylate (and) PPG-1 Trideceth-6, Polyquaternium-7, Polyquaternium-44.

### Emulsifiers

Suitable emulsifiers are, for example, nonionogenic surfactants from at least one of the following groups:
addition products of from 2 to 30 mol of ethylene oxide and/or 0 to 5 mol of propylene oxide onto linear fatty alcohols having, 8 to 22 carbon atoms, onto fatty acids having 12 to 22 carbon atoms and onto alkylphenols having 8 to 15 carbon atoms in the alkyl group;
C12/18 fatty acid mono- and diesters of addition products of from 1 to 30 mol of ethylene oxide onto glycerol;
glycerol mono- and diesters and sorbitan mono- and diesters of saturated and unsaturated fatty acids having 6 to 22 carbon atoms and ethylene oxide addition products thereof;
alkyl mono- and oligoglycosides having 8 to 22 carbon atoms in the alkyl radical and ethoxylated analogs thereof;
addition products of from 15 to 60 mol of ethylene oxide onto castor oil and/or hardened castor oil;
polyol and, in particular, polyglycerol esters, such as, for example, polyglycerol polyricinoleate, polyglycerol poly-12-hydroxy stearate or polyglycerol dimerate. Mixtures of compounds from two or more of these classes of substances are likewise suitable;
addition products of from 2 to 15 mol of ethylene oxide onto castor oil and/or hardened castor oil;
partial esters based on linear, branched, unsaturated or saturated C_{6/22}-fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose);
mono-, di- and trialkyl phosphates, and mono-, di- and/or tri-PEG alkyl phosphates and salts thereof;
wool wax alcohols;
polysiloxane-polyalkyl-polyether copolymers and corresponding derivatives;
mixed esters of pentaerythritol, fatty acids, citric acid and fatty alcohol as in DE-C 1165574 and/or mixed esters of fatty acids having 6 to 22 carbon atoms, methylglycose and polyols, preferably glycerol or polyglycerol, and
polyalkylene glycols.

The addition products of ethylene oxide and/or of propylene oxide onto fatty alcohols, fatty acids, alkylphenols, glycerol mono- and diesters, and sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C₁₂ to C₁₈-fatty acid mono- and diesters of addition products of ethylene oxide onto glycerol are known from DE-C 2024051 as refatting agents for cosmetic preparations. C₈ to C₁₈-alkyl mono- and oligoglycosides, their preparation and their use are known from the prior art. Their preparation takes place in particular by reacting glucose or oligosaccharides with primary alcohols having 8 to 18 carbon atoms. With regard to the glycoside ester, either monoglycosides, in which a cyclic sugar radical is bonded glycosidically to the fatty alcohol, or oligomeric glycosides with a degree of oligomerization up to preferably about 8 are suitable. The degree of oligomerization here is a statistical mean value which is based on a homolog distribution customary for such technical products.

Furthermore, zwitterionic surfactants can be used as emulsifiers. Zwitterionic surfactants is the term used to refer to those surface-active compounds which carry at least one quaternary ammonium group and at least one carboxylate group and/or one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocosacylaminopropyldimethylammonium glycinate, and 2-alkyl-3-carboxylmethyl-3-hydroxyethylimidazolines having in each case 8 to 18 carbon atoms in the alkyl or acyl group, and cocosacylaminoethyl hydroxyethylcarboxymethyl glycinate.

Particular preference is given to the fatty acid amide derivative known under the CTFA name Cocamidopropyl Betaine. Likewise suitable emulsifiers are ampholytic surfactants. Ampholytic surfactants are understood as meaning those surface-active compounds which, apart from a C₈ to C₁₈-alkyl or acyl group in the molecule, comprise at least one free amino group and at least one —COOH and/or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocosalkylaminopropionate, cocosacylaminoethylaminopropionate and C₁₂ to C₁₈-acylsarcosine.

Besides the ampholytic emulsifiers, quaternary emulsifiers are also suitable, particular preference being given to those of the ester quat type, preferably methyl-quaternized difatty acid triethanolamine ester salts.

### Antioxidants

An additional content of antioxidants in the preparations may be advantageous. According to the invention, antioxidants which may be used are all antioxidants customary or suitable for cosmetic applications. The antioxidants are advantageously selected from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, γ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylidene glucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

The amount of the abovementioned antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to provide these in concentrations of from 0.001 to 10% by weight, based on the total weight of the preparation.

If vitamin A or vitamin A derivatives, or carotenes or derivatives thereof are the antioxidant or the antioxidants, it is advantageous to provide these in concentrations of from 0.001 to 10% by weight, based on the total weight of the preparation.

### Perfume Oils

The cosmetic, preferably hair cosmetic, preparations can comprise perfume oils. Perfume oils which may be mentioned are, for example, mixtures of natural and synthetic fragrances. Natural fragrances are extracts of flowers (lily, lavender, rose, jasmine, neroli, ylang-ylang), stems and leaves (geranium, patchouli, petitgrain), fruits (anise, coriander, caraway, juniper), fruit peels (bergamot, lemon, orange), roots (mace, angelica, celery, cardamon, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Animal raw materials are also suitable, such as, for example, cibet and castoreum. Typical synthetic fragrance compounds are products of the ester type, ether type, aldehyde type, ketone type, alcohol type and hydrocarbon type. Fragrance compounds of the ester type are, for example, benzyl acetate, phenoxyethyl isobutyrate, 4-tert-butyl cyclohexylacetate, linalylacetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether; the aldehydes include, for example, the linear alkanals having 8 to 18 carbon atoms, citral, citronellal, citronellyloxyacetaldehyde, cyclamenaldehyde, hydroxycitronellal, filial and bourgeonal, the ketones include, for example, the ionones, cc-isomethylionene and methyl cedryl ketone, the alcohols include anethol, citronellol, eugenol, isoeugenol, geraniol, linalool, phenylethyl alcohol and terioneol, the hydrocarbons include primarily the terpenes and balsams. However, preference is given to using mixtures of different fragrances which together produce a pleasant scent note. Essential oils of relatively low volatility, which in most cases are used as aroma components, are also suitable as perfume oils, e.g. sage oil, camomile oil, clove oil, melissa oil, mint oil, cinnamon leaf oil, linden blossom oil, juniper berry oil, vetiver oil, olibanum oil, galbanum oil, labolanum oil and lavendin oil. Preference is given to using bergamot oil, dihydromyrcenol, lilial, lyral, citronellol, phenylethyl alcohol, a-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clay sage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat, alone or in mixtures.

### Superfatting Agents

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter serving simultaneously as foam stabilizers.

### Silicone Compounds

In one embodiment, the preparations according to the invention comprise, as hair care additive, at least one silicone compound in an amount of from preferably 0.01 to 15% by weight, particularly preferably from 0.1 to 5% by weight. The silicone compounds comprise volatile and nonvolatile silicones and silicones that are insoluble or soluble in the composition. In one embodiment, these are high molecular weight silicones with a viscosity of from 1000 to 2 000 000 cSt at 25° C., preferably 10 000 to 1 800 000 or 100 000 to 1 500 000. The silicone compounds comprise polyalkyl- and polyarylsiloxanes, in particular with methyl, ethyl, propyl, phenyl, methylphenyl and phenylmethyl groups. Preference is, given to polydimethylsiloxanes, polydiethylsiloxanes, polymethylphenylsiloxanes. Preference is also given to luster-imparting, arylated silicones with a refractive index of at, least 1.46, or at least 1.52. The silicone compounds comprise, in particular, the substances with the INCI names Cyclomethicone, Dimethicone, Dimethiconol, Dimethicone Copolyol, Phenyl Trimethicone, Amodimethicone, Trimethylsilylamodimethicone, Stearyl Siloxysilicate, Polymethylsilsesquioxane, Dimethicone Crosspolymer. Also suitable are silicone resins and silicone elastomers, which are highly crosslinked siloxanes.

Preferred silicones are cyclic dimethylsiloxanes, linear polydimethylsiloxanes, block polymers of polydimethylsiloxane and polyethylene oxide and/or polypropylene oxide, polydimethylsiloxanes with terminal or lateral polyethylene oxide or polypropylene oxide radicals, polydimethylsiloxanes with terminal hydroxyl groups, phenyl-substituted polydimethylsiloxanes, silicone emulsions, silicone elastomers, silicone waxes, silicone gums and amino-substituted silicones (CTFA: Amodimethicone).

### Hair Conditioners

In one embodiment, the preparations according to the invention comprise 0.01 to 20% by weight, preferably from 0.05 to 10% by weight, particularly preferably from 0.1 to 5% by weight, of at least one conditioner.

Conditioners preferred according to the invention are, for example, all compounds which are listed in the International Cosmetic. Ingredient Dictionary and Handbook (Volume 4, editor: R. C. Pepe, J. A. Wenninger, G. N. McEwen, The Cosmetic, Toiletry, and Fragrance Association, 9th edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all compounds listed in EP-A 934 956 (pp. 11-13) under “water soluble conditioning agent” and “oil soluble conditioning agent”. Further advantageous conditioners are, for example, the compounds referred to in accordance with INCI as Polyquatemium (in particular Polyquaternium-1 to Polyquaternium-56).

Suitable conditioners include, for example, also polymeric quaternary ammonium compounds, cationic cellulose derivatives, chitosan derivatives and polysaccharides. The conditioner is preferably selected from betaine, panthenol, panthenyl ethyl ether, sorbitol, protein hydrolyzates, plant extracts; A-B block copolymers of alkyl acrylates and alkyl methacrylates; A-B block copolymers of alkyl methacrylates and acrylonitrile; A-B-A block copolymers of lactide and ethylene oxide; A-B-A block copolymers of caprolactone and ethylene oxide; A-B-C block copolymers of alkylene and alkadiene compounds, styrene and alkyl methacrylates; A-B-C block copolymers of acrylic acid, styrene and alkyl methacrylates, stellate block copolymers, hyperbranched polymers, dendrimers, intrinsically electrically conductive 3,4-polyethylenedioxythiophenes and intrinsically electrically conductive polyanilines.

Further conditioners advantageous according to the invention are cellulose derivatives and quaternized guar gum derivatives, in particular guar hydroxypropylammonium chloride (e.g. Jaguar Excel®, Jaguar C 162® (Rhodia), CAS 65497-29-2, CAS 39421-75-5).

Nonionic poly-N-vinylpyrrolidone/polyvinyl acetate copolymers (e.g. Luviskol®VA 64 (BASF)), anionic acrylate copolymers (e.g. Luviflex®Soft (BASF)), and/or amphoteric amide/acrylate/methacrylate copolymers (e.g. Amphomer® (National Starch)) can also be used advantageously according to the invention as conditioners.

### Hydrotropes

To improve the flow behavior, hydrotropes, such as, for example, ethanol, isopropyl alcohol, or polyols, can also be used. Polyols which are of suitability here preferably have 2 to 15 carbon atoms and at least two hydroxyl groups. Typical examples are
glycerol;
alkylene glycols, such as, for example, ethylene glycol, diethylene glycol, propylene glycol, butylene glycol, hexylene glycol, and polyethylene glycols with an average molecular weight of from 100 to 1000 daltons;
technical-grade oligoglycerol mixtures with a degree of self-condensation of from 1.5 to 10, such as, for example, technical-grade diglycerol mixtures with a diglycerol content of from 40 to 50% by weight;
methylol compounds, such as, in particular, trimethylolethane, trimethylolpropane, trimethylolbutane, pentaerythritol and dipentaerythritol;
lower alkyl glucosides, in particular those having 1 to 8 carbon atoms in the alkyl radical, such as, for example, methyl and butyl glucoside;
sugar alcohols having 5 to 12 carbon atoms, such as, for example, sorbitol or mannitol;
sugars having 5 to 12 carbon atoms, such as, for example, glucose or sucrose;
amino sugars, such as, for example, glucamine.

### Oils, Fats and Waxes

The cosmetic, preferably hair cosmetic, preparations according to the invention can also comprise oils, fats or waxes. These are advantageously selected from the group of lecithins and fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, be selected advantageously from the group of synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and the like. Further polar oil components can be selected from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, as well as from the group of esters of aromatic carboxylic acids and saturated and/or unsaturated, branched and/or unbranched alcohols of chain length from 3 to 30 carbon atoms. Such ester oils can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (Myritol 331), butylene glycol dicaprylate/dicaprate and dibutyl adipate, and synthetic, semisynthetic and natural mixtures of such esters, such as, for example, jojoba oil.

Furthermore, one or more oil components can advantageously be selected from the group of branched and unbranched hydrocarbons and hydrocarbon waxes, of silicone oils, of dialkyl ethers, the group of saturated or unsaturated, branched or unbranched alcohols.

Any mixtures, of such oil and wax components are also to be used advantageously for the purposes of the present invention. It may also, if appropriate, be advantageous to use waxes, for example cetyl palmitate, as the sole lipid component of the oil phase. According to the invention, the oil component is advantageously selected from the group 2-ethylhexyl isostearate, octyldodecanol, isotridecyl isononanoate, isoeicosane, 2-ethylhexyl cocoate, C12-15-alkyl benzoate, caprylic/capric triglyceride, dicaprylyl ether.

According to the invention, mixtures of C12-15-alkylbenzoate and 2-ethylhexyl isostearate, mixtures of C12-15-alkylbenzoate and isotridecyl isononanoate, and also mixtures of C12-15-alkylbenzoate, 2-ethylhexyl isostearate and isotridecyl isononanoate are advantageous.

According to the invention, the oils with a polarity of from 5 to 50 mN/m used are particularly preferably fatty acid triglycerides, in particular soybean oil and/or almond oil.

In addition, the oil phase can advantageously be selected from the group of Guerbet alcohols. These are liquid even at low temperatures and cause virtually no skin irritations. They can advantageously be used as fatting, superfatting and also refatting constituents in cosmetic compositions.

The use of Guerbet alcohols in cosmetics is known per se. Guerbet alcohols preferred according to the invention are 2-butyloctanol (commercially available, for example, as Isofol® 12 (Condea)) and 2-hexyldecanol (commercially available, for example, as Isofol® 16 (Condea)).

Mixtures of Guerbet alcohols according to the invention are also to be used advantageously according to the invention, such as, for example, mixtures of 2-butyloctanol and 2-hexyldecanol (commercially available, for example, as Isofol® 14 (Condea)).

Any mixtures of such oil and wax components are also to be used advantageously for the purposes of the present invention. Among the polyolefins, polydecenes are the preferred substances.

Fat and/or wax components to be used advantageously according to the invention can be selected from the group of vegetable waxes, animal waxes, mineral waxes and petrochemical waxes. For example, candelilla wax, carnauba wax, Japan wax, espartograss wax, cork wax, guaruma wax, rice germ oil wax, sugar cane wax, berry wax, ouricury wax, montan wax, jojoba wax; shea butter, beeswax, schellack wax, spermaceti, lanolin (wool wax), uropygial grease, ceresin, ozokerite (earth wax), paraffin waxes and microwaxes are advantageous.

Further advantageous fat and/or wax components are chemically modified waxes and synthetic waxes, such as, for example, Syncrowax®HRC (glyceryl tribehenate), and Syncrowax®AW 1 C(C₁₈₋₃₆-fatty acid), and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates, such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkylhydroxystearoyl stearate and/or glycol montanate. Further advantageous are also certain organosilicon compounds which have similar physical properties to the specified fat and/or wax components, such as, for example, stearoxytrimethylsilane. According to the invention, the fat and/or wax components can be used either individually or else as a mixture in the compositions.

Any mixtures of such oil and wax components are also to be used advantageously for the purposes of the present invention.

The oil phase is advantageously selected from the group 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric acid triglyceride, dicaprylyl ether.

Mixtures of octyldodecanol, caprylic/capric triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are to be used advantageously for the purposes of the present invention.

The oil component is also advantageously selected from the group of phospholipids. According to the invention, Merkur®Weissoel Pharma 40 from Merkur Vaseline, Shell Ondina® 917, Shell Ondina® 927, Shell Oil 4222, Shell Ondina®933 from Shell & DEA Oil, Pionier® 6301 S, Pionier® 2071 (Hansen & Rosenthal) can be used as paraffin oil advantageous according to the invention.

Suitable cosmetically compatible oil and fat components are described in Karl-Heinz Schrader, Grundlagen and Rezepturen der Kosmetika [Fundamentals and Formulations of Cosmetics], 2nd edition, Verlag Hüthig, Heidelberg, pp. 319-355, to which reference is hereby made in its entirety.

The content of oils, fats and waxes is at most 30% by weight, preferably 20% by weight, further preferably at most 10% by weight, based on the total weight of the composition.

### Pigments

In one embodiment, the preparations according to the invention comprise at least one pigment. These may be colored pigments which impart color effects to the product mass or to the hair, or they may be luster effect pigments which impart luster effects to the product mass or to the hair. The color or luster effects on the hair are preferably temporary, i.e. they remain on the hair until the next wash and can be removed again by washing the hair with customary shampoos.

The pigments are present in the product mass in undissolved form and may be present in an amount of from 0.01 to 25% by weight, particularly preferably from 5 to 15% by weight. The preferred particle size is 1 to 200 μm, in particular 3 to 150 μm, particularly preferably 10 to 100 μm. The pigments are colorants that are virtually insoluble in the application medium and may be inorganic or organic. Inorganic-organic mixed pigments are also possible. Preference is given to inorganic pigments. The advantage of inorganic pigments is their excellent resistance to light, weather and temperature. The inorganic pigments may be of natural origin, for example produced from chalk, ocher, umber, green earth, burnt siena or graphite. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescence or phosphorescence pigments, where preferably at least one pigment is a colored, non-white pigment.

Metal oxides, hydroxides and oxide hydrates, mixed-phase pigments, sulfur-containing silicates, metal sulfides, complex metal cyanides, metal sulfates, chromates and molybdates, and the metals themselves (bronze pigments) are suitable. In particular, titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI77289), iron blue (ferric ferrocyanide, CI7751 0), carmine (cochineal) are suitable.

Particular preference is given to pearlescent and colored pigments based on mica which have been coated with a metal oxide or a metal oxychloride, such as titanium dioxide or bismuth oxychloride, and, if appropriate; further color-imparting substances such as iron oxides, iron blue, ultramarine, carmine etc., and where the color can be determined by varying the layer thickness. Such pigments are sold; for example, under the trade names Rona®, Colorona®, Dichrona® and Timiron® by Merck, Germany.

Organic pigments are, for example, the natural pigments sepia, gamboge, bone charcoal, cassel brown, indigo, chlorophyll and other plant pigments. Synthetic organic pigments are, for example, azo pigments, anthraquinoids, indigoids, dioxazin, quinacridone, phthalocyanine, isoindolinone, perylene and perinone pigments, metal complex pigments, alkali blue and diketopyrrolopyrrole pigments.

In one embodiment, the preparations according to the invention comprise 0.01 to 10% by weight, particularly preferably from 0.05 to 5% by weight, of at least one particulate substance. Suitable substances are, for example, substances which are solid at room temperature (25° C.) and are in the form of particles. Of suitability are, for example, silica, silicates, aluminates, clay earths, mica, salts, in particular inorganic metal salts, metal oxides, e.g. titanium dioxide, minerals and polymer particles.

The particles are present in the composition in undissolved, preferably stably dispersed form and, following application to the application surface and evaporation of the solvent, can deposit in solid form.

Preferred particulate substances are silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

Suitable repellent active ingredients are compounds which are able to keep away or to drive away certain animals, in particular insects; from humans. These include, for example, 2-ethyl-1,3-hexanediol, N,N-diethyl-m-toluamide etc. Suitable hyperemic substances, which stimulate blood flow through the skin, are, for example, essential oils, such as dwarf-pine, lavender, rosemary, juniper berry, horse chestnut extract, birch leaf extract, hay flower extract, ethyl acetate, camphor, menthol, peppermint oil, rosemary extract, eucalyptus oil, etc.

Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc.

Suitable antiphlogistics, which counteract skin irritations, are, for example, allantoin, bisabolol, dragosantol, camomile extract, panthenol, etc.

### Administration Form

In a preferred embodiment, the preparations according to the invention are sprayable, for example as aerosol or pumpspray preparation.

The preparations according to the invention can be applied in various forms, such as, for example, as lotion, as nonaerosol spray lotion, which is used by means of a mechanical device for spraying, as aerosol spray which is sprayed by means of a propellant, as aerosol foam or as nonaerosol foam, which is present in combination with a suitable mechanical device for foaming the composition, as hair cream, as hair wax, as gel, as liquid gel, as sprayable gel or as foam gel.

Use in the form of a lotion thickened with a customary thickener is also possible.

In one embodiment, the composition according to the invention is in the form of a gel, in the form of a viscous lotion or in the form of a spray gel, which is sprayed with a mechanical device, and comprises at least one of the abovementioned thickeners in an amount of from preferably 0.05 to 10% by weight, particularly preferably from 0.1 to 2% by weight and has a viscosity of at least 250 mPas. The viscosity of the gel is preferably from 500 to 50 000 mPas, particularly preferably from 1000 to 15 000 mPas at 25° C.

In another embodiment, the preparation according to the invention is in the form of an O/W emulsion, a W/O emulsion or a microemulsion and comprises at least one of the abovementioned, water-emulsified oils or waxes, and at least one cosmetically customary surfactant.

In a preferred embodiment, the preparation according to the invention is in the form of a spray product, either in combination with a mechanical pump spray device or in combination with at least one of the abovementioned propellants. A preferred aerosol spray additionally comprises propellants in an amount such that the total amount of the volatile organic components does not exceed 80% by weight, in particular 55% by weight, of the preparation and is bottled in a pressurized container.

A nonaerosol hair spay is sprayed with the help of a suitable mechanically operated spray device. Mechanical spray devices are to be understood as meaning those devices which permit the spraying of a composition without use of a propellant. A suitable mechanical spray device which may be used is, for example, a spray pump or an elastic container provided with a spray valve and into which the cosmetic preparation according to the invention is bottled under pressure, where the elastic container expands and from which the composition is continuously dispensed as a result of the contraction of the elastic container upon opening the spray valve.

In a further embodiment, the preparation according to the invention is in the form of a foamable product (mousse) in combination with a device for foaming, comprises at least one customary foam-imparting substance known for this purpose, for example at least one foam-forming surfactant or at least one foam-forming polymer. Devices for foaming are to be understood as meaning those devices which permit the foaming of a liquid with or without use of a propellant. A suitable mechanical foam device which can be used is, for example, a standard commercial pump foamer or an aerosol foam head. The product is present either in combination with a mechanical pump foam device (pump foam) or in combination with at least one propellant (aerosol foam) in an amount of from preferably 1 to 20% by weight, in particular from 2 to 10% by weight. Propellants are, for example, selected from propane, butane, dimethyl ether and fluorinated hydrocarbons.

The invention thus provides a cosmetic, preferably hair cosmetic, preparation in the form of a spray product, where the preparation is present either in combination with a mechanical pump spray device or in combination with at least one propellant selected from the group consisting of propane, butane, dimethyl ether, fluorinated hydrocarbons and mixtures thereof.

The composition is foamed directly prior to application and is incorporated into the hair in the form of a foam and can then be rinsed out or left in the hair without rinsing.

A formulation for aerosol hair foams preferred according to the invention comprises
i) 0.1 to 10% by weight of at least one polymer A,
ii) 55 to 99.8% by weight of water and alcohol,
iii) 5 to 20% by weight of a propellant,
iv) 0.1 to 5% by weight of an emulsifier,
v) 0 to 10% by weight of further constituents,
where the total amount of VOC is at most 80% by weight and preferably 55% by weight.

Emulsifiers which can be used are all emulsifiers customarily used in hair foams. Suitable emulsifiers may be nonionic, cationic or anionic or amphoteric.

Examples of nonionic emulsifiers (INCI nomenclature) are laureths, e.g. laureth-4; ceteths, e.g. ceteth-1, polyethylene glycol cetyl ether; ceteareths, e.g. cetheareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimoniummethylsulfate, quaternium-1 to x (INCI).

Anionic emulsifiers can, for example, be selected from the group of alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
i) 0.1 to 10% by weight of polymer A,
ii) 80 to 99.85% by weight of water and alcohol,
iii) 0 to 3% by weight, preferably 0.05 to 2% by weight, of a gel former,
iv) 0 to 20% by weight of further constituents
where the total amount of VOC is at most 80% by weight and preferably 55% by weight.

In the preparation of gels based on the polymers A, customary gel formers can be used, for example in order to establish specific rheological or other application properties of the gels. Gel formers which can be used are all gel formers customary in cosmetics. These include lightly crosslinked polyacrylic acid, for example carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) Paraffinum Liquidum (and) PPG-1 trideceth-6, acrylamidopropyltrimonium chloride/acrylamide copolymers, steareth-10 alkyl ether acrylate copolymers, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44. Crosslinked homopolymers of acrylic acid suitable as gel formers are commercially available, for example, under the name Carbopol® (Noveon). Preference is also given to hydrophobically modified crosslinked polyacrylate polymers, such as Carbopol®Ultrez 21 (Noveon). Further examples of anionic polymers suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are commercially available as Aculyn® (Rohm and Haas).

In a further embodiment, the preparation according to the invention is in the form of a hair wax, i.e. it has wax-like consistency and comprises at least one of the abovementioned waxes in an amount of from preferably 0.5 to 30% by weight, and, if appropriate, further water-insoluble substances. The wax-like consistency is preferably characterized in that the needle penetration number (measurement unit 0.1 mm, test weight 100 g, test time 5 s, test temperature 25° C.; in accordance with DIN 51 579) is greater than or equal to 10, particularly preferably greater than or equal to 20, and that the solidification point of the product is preferably greater than or equal to 30° C. and less than or equal to 70° C., particularly preferably in the range from 40 to 55° C. Suitable waxes and water-insoluble substances are, in particular, emulsifiers with an HLB value below 7, silicone oils, silicone waxes, waxes: (e.g. wax alcohols, wax acids, wax esters, and in particular natural waxes, such as beeswax, carnauba wax, etc), fatty alcohols, fatty acids, fatty acid esters or hydrophilic waxes, such as, for example, high molecular weight polyethylene glycols with a molecular weight of from 800 to 20 000 g/mol, preferably from 2000 to 10 000 g/mol.

If the cosmetic, preferably hair cosmetic, preparation according to the invention is in the form of a hair lotion, then it is in the form of an essentially nonviscous or low-viscosity, flowable solution, dispersion or emulsion with a content of at least 10% by weight, preferably 20 to 95% by weight, of a cosmetically compatible alcohol. Alcohols which can be used are, in particular, the lower alcohols having 1 to 4 carbon atoms customarily used for cosmetic purposes, for example ethanol and isopropanol.

If the hair cosmetic preparation according to the invention is in the form of a hair cream, then it is preferably in the form of an emulsion and comprises either additionally viscosity-imparting ingredients in an amount of from 0.1 to 10% by weight, or the required viscosity and creamy consistency is built up by micelle formation with the help of suitable emulsifiers, fatty acids, fatty alcohols, waxes etc in the customary manner.

The polymers A according to the invention can be used in cosmetic preparations as conditioners.

The polymers A according to the invention can preferably be used in shampoo formulations as setting agents and/or conditioners. Preferred shampoo formulations comprise
i) 0.05 to 10% by weight of at least one polymer. A,
ii) 25 to 94.95% by weight of water,
iii) 5 to 50% by weight of surfactants,
iv) 0 to 5% by weight of a further conditioner,
v) 0 to 10% by weight of further cosmetic constituents.

In the shampoo formulations it is possible to use all anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate triethanolamine dodecylbenzenesulfonate are suitable.

Suitable amphoteric surfactants are, for example, alkylbetaines, alkylamidopropylbetaines, alkylsulfobetaines, alkyl glycinates, alkyl carboxyglycinates, alkyl amphoacetates or propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, alkyl polyglycosides or sorbitan ether esters.

Furthermore, the shampoo formulations can comprise customary cationic surfactants, such as, for example, quaternary ammonium compounds, for example cetyltrimethylammonium chloride.

In the shampoo formulations, to achieve certain effects, customary conditioners can be used in combination with the polymers. A. These include, for example, the abovementioned cationic polymers with the INCI name Polyquatemium, in particular copolymers of vinylpyrrolidone/N-vinylimidazolium salts (Luviquat®FC, Luviquat®HM, Luviquat®MS, Luviquat®Care, Luviquat®Ultracare), copolymers of N-vinylpyrrolidone/dimethylaminoethyl methacrylate, quaternized with diethyl sulfate (Luviquat®PQ 11), copolymers of N-vinylcaprolactam/N-vinylpyrrolidone/N-vinylimidazolium salts (Luviquat®Hold); cationic cellulose derivatives (Polyquaternium-4 and -10), acrylamide copolymers (Polyquaternium-7). Furthermore, protein hydrolyzates can be used, as can conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino-functional silicone compounds, such as amodimethicones (CTFA). In addition, cationic guar derivatives, such as guar hydroxypropyltrimonium chloride (INCI) can be used.

### Measurement Methods

### Determination of the K Value

The K values are measured in accordance with Fikentscher, Cellulosechemie [Cellulose Chemistry], Vol. 13, pp. 58 to 64 (1932) at 25° C. in ethanol or N-methylpyrrolidone (NMP) solution and are a measure of the molecular weight. The ethanol or NMP solutions of the polymers in each case comprise 1 g of polymer A in 100 ml of solution.

If the polymers are in the form of aqueous dispersions, corresponding amounts of the dispersion, depending on the polymer content of the dispersion, are made up to 100 ml with ethanol such that the concentration is 1 g in 100 ml.

The K value is measured in a micro-Ubbelohde capillary type M Ic from Schott.

Determination of the particle size distribution (PSD) by means of Malvern® scattered-light analysis.

The particle size distribution was determined using a particle size measurement system for detecting liquid aerosols “Malvern® Master Sizer X” (Malvern Instruments Inc., Southborough Mass., USA).

### Measurement Principle:

The measurement system is based on the method of laser light diffraction at the particle which, apart from being suitable for the spray analysis (aerosols, pump sprays), is also suitable for the size determination of solids, suspensions and emulsions in the size range from 0.1 μm to 2000 μm.

A particle collective (=droplet) is illuminated by a laser. At each droplet, some of the incident laser light is scattered. This light is captured on a multielement detector and the associated light energy distribution is determined. Using the evaluation software, the associated particle distribution is calculated from this data.

### Procedure:

The aerosols were sprayed in at a distance of 29.5 cm from the laser beam. The spray cone entered perpendicularly relative to the laser beam.

Prior to each measurement, the aerosol cans were fixed to a firmly installed holding device, thus ensuring that all of the aerosols to be tested were measured at exactly the same distance.

Before the actual particle measurement, a “background measurement” was carried out. In doing this, the effects of dust and other contaminants in the measurement range were eliminated.

The aerosol was then sprayed into the test space. The total particle volume was ascertained over a test period of 2 s and evaluated.

### Evaluation:

The evaluation comprises a tabular representation over 32 class widths from 0.5 μm to 2000 μm and additionally a graphical representation of the particle size distribution.

Since the distribution in the spray experiments is approximately uniform, the mean diameter D(v, 0.5) is given. This numerical value indicates that 50% of the particle volume measured overall is below this value.

For readily sprayable aerosol systems in the cosmetic sector, this value is in the range from 30 μm to 80 μm depending on polymer content, valve and sprayhead geometry, solvent ratio and amounts of propellant gas.

### Determination of the Setting (Flexural Rigidity):

The setting of polymeric film formers was measured, apart from by subjective assessment (hand test), also physically by measuring the flexural rigidity of thin hair tresses (in each case about 3 g and 24 cm in length). For this, the weighed, dry hair tresses were dipped into the 3.0% strength by weight polymer solution (solvent: ethanol/water 55:45 w/w), uniform wetting of the hair tress and distribution of the polymer solution being ensured through triple immersion and removal and subsequent squeezing between filter paper. The excess film former solution was then stripped off between thumb and forefinger and the hair tresses were shaped by hand in such a way that they adopted a round cross section. Drying was carried out at 20° C. and 65% relative humidity overnight in a climatically controlled room. The tests were carried out in the climatically controlled room at 20° C. and 65% relative humidity using a stress/strain testing device. The hair tress was placed symmetrically at the ends on two cylindrical rolls of the sample holder. The tress was then bent exactly in the middle from above using a rounded punch ca. 40 mm (fracture of the polymer film). The force required for this (Fmax) was determined using a weighing cell (50 N). Here, a measurement value constitutes the arithmetic mean from the individual measurements on 5 to 10 equally treated hair tresses. The values ascertained in this way were compared to those of a standard commercial comparison polymer (Amphomer®LV-71) and given in %.

### Determination of the Ability to be Washed Out

A hair tress treated analogously to the determination of the setting with polymer was washed in an approximately 37° C.-hot Texapon®NSO solution (6 ml of Texapon®NSO (28% strength) in 1 l of warm water) for ca. 15 seconds by immersion and squeezing 5 times. The hair tress was then rinsed until clear and treated again in the same way. The hair tress was then squeezed well on filter paper and left to dry overnight. The dry hair tress was wound and investigated for residues.

### Determination of the Curl Retention

### Base Formulation: (Aerosol Hair Spray)

| | |
|---|---|
| 5% by wt. | active ingredient of polymer to be tested (100% neutr. with |
| | AMP) |
| 15% by wt. | ethanol |
| 40% by wt. | water |
| 40% by wt. | dimethyl ether. |

For the determination of curl retention, hair tresses ca. 2 g in weight and 15.5 cm in length and made of mid-brown, Caucasian human hair were used.

### Treatment of the Hair Tresses:

The hair tresses were washed twice with an aqueous Texapon®NSO solution. The hair tresses were then rinsed with warm water until foam formation could no longer be detected, and rinsed with demineralized water, combed and laid to dry on filter paper.

To produce a water wave, the hair tresses are placed in a solution of ethanol and water (0.1:1) for swelling for 15 minutes.

The hair tress was carefully combed prior to curl preparation. A rubber band was used to attach the hair tress to the Plexiglass rod. The hair was then combed and wound in the form of a spiral. Using a cotton cloth and rubber band, the curl was firmly fixed and dried overnight at 70° C. The cooled curl retention tresses were carefully opened and stripped off from the Plexiglass rod without deforming the water wave. From a distance of 15 cm, 1.8 g of the aerosol hair spray produced as above were sprayed evenly onto the curl. The curl was rotated evenly during this time. In a horizontal position, the curls were dried for 1 h at room temperature. After drying, the curls were fastened in a holder. Using a ruler, at the start, the initial length of the curls was read off and the length extension was monitored during the humidity storage. After storage for 5 h at 25° C. and 90% relative humidity in the climatically controlled chamber, the length of the curl achieved was read off again and the curl retention was calculated according to the following equation:

$$Curl Retention in \% = \frac{L - {L}_{t}}{L - {L}_{o}} \times 100$$

L=Length of the hair (15.5 cm).

L₀=Length of the hair curl after drying

Lₜ=Length of the hair curl after humidity treatment

The mean value from the 5 individual measurements was stated as curl retention.

### Determination of the Stickiness

A clear, 20% strength by weight ethanolic or ethanolic/aqueous solution of the polymer to be characterized was firstly produced. In order to obtain a clear solution, it was sometimes necessary to neutralize the polymer. A film of the polymer was then applied to a glass plate from the ethanolic or ethanolic/aqueous solution using a doctor knife (gap width 120 μm). This rectangular glass plate had a length of ca. 20 cm and a width of ca. 6.5 m. The polymer film applied thereto in each case had a length of ca. 16 to 18 cm and a width of ca. 5.5 cm.

The film was then dried in the air for ca. 10 hours and then stored in a climatically controlled cabinet at 20° C. and 80% relative humidity for a further 12 hours.

Under these conditions, a plastic carbon ribbon (e.g. Pelikan®2060, 50 mm wide) located on a round rubber stamp (diameter 400 mm, Shore A hardness 60±5) was then pressed onto the polymer film in the climatically controlled cabinet with a force of ca. 250 N for 10 seconds.

The amount of the black pigment which remains stuck to the polymer film after removing the stamp corresponds to the stickiness of the film. A visual assessment of the black coloration of the film was made. The assessment scale ranges from 0 to 5, where 0 is not sticky and 5 is very sticky.

### Determination of the Appearance of the Aerosol Formulation

The preparation comprising 5% by weight of the particular polymer neutralized with AMP, 40% by weight of DME, 15% by weight of ethanol and 40% by weight of water was poured into a transparent glass aerosol container. The clarity of the resulting liquid/propellant gas mixture was then assessed visually.

### EXAMPLES

The examples below illustrate the invention without limiting it thereto. Unless designated otherwise, the percentages are % by weight.

### Abbreviations Used:

MAA methacrylic acid

AA acrylic acid

MMA methyl methacrylate

Laromer® BDDA butanediol diacrylate

Laromer® TPGDA tripropylene glycol diacrylate

DEM. water demineralized water

| Example | Monomers | Ratio |
|---|---|---|
| C1 | MMA/AA | 75/25 |
| C2 | MMA/MAA | 75/25 |
| C3 | MMA/MAA/Laromer ® BDDA | 73/25/2 |
| 1 | MMA/MAA/AA | 75/15/10 |
| 2 | MMA/MAA/AA/Laromer ® TPGDA | 74/15/10/1 |
| 3 | MMA/MAA/AA | 80/10/10 |
| 4 | MMA/MAA/AA | 80/10/10 |
| 5 | MMA/MAA/AA/Laromer ® BDDA | 78/10/10/2 |
| 6 | MMA/MAA/AA | 75/20/5 |
| 7 | MMA/MAA/AA | 80/10/10 |

### Preparation of Comparative Example 1 (C1)

The following feeds were prepared with stirring at 20° C.:

Feed 1

| | |
|---|---|
| 111 g | MMA |
| 37.5 g | AA |
| 100 g | Ethanol |

Feed 2

| | |
|---|---|
| 1.5 g | Wako ® V 59 |
| 50 g | Ethanol |

A mixture of 150 g of ethanol, 15% by weight of the total amount of feed 1, and 15% by weight of the total amount of feed 2 was prepared at 20° C. The mixture was heated to 78° C. under atmospheric pressure. While retaining the polymerization temperature, feeds 1 and 2 were started after reaching 78° C. Feed 1 was metered in over the course of 3 hours, and feed 2 was metered in over the course of 4 hours with constant feed stream. When feed 2 was complete, the reaction mixture was kept at 78° C. for a further 2 h and then cooled to room temperature (ca. 20° C.).

### Preparation of Comparative Example 2 (C2)

At a temperature of 20 to 25° C.,

| | |
|---|---|
| 400 g of | deionized water |
| 0.6 g of | 15% strength by weight aqueous solution of sodium |
| | lauryl sulfate in deionized water |
| 35 g of | feed II (see below) |

were initially introduced into a 2 l polymerization vessel with stirrer and heating and cooling devices and heated to 45° C. with stirring and under a nitrogen atmosphere. After reaching this temperature, I feed was added over the course of 5 minutes. The mixture was then heated to 80° C. and, with stirring and while retaining the reaction temperature, feed II was metered in over the course of 2.5 hours with constant feed streams. When the feeds were complete, the reaction mixture was stirred for a further hour at 80° C. and then cooled to 60°. While maintaining the temperature of 60°, feed III was added. The mixture was then cooled to 35° C. and, while retaining the reaction temperature, feed IV was added.

### Feed I:

| | |
|---|---|
| 6 g | 7% strength by weight aqueous solution of sodium persulfate |
| | in deionized water |

Feed II is an Aqueous Monomer Emulsion Prepared from:

| | | % by wt. based on the |
|---|---|---|
| Initial weight | | total amount of monomer |
| 204 g | deionized water | |
| 8 g | 15% strength by weight | |
| | solution of sodium lauryl | |
| | sulfate in deionized water | |
| 10 g | Tween ™ 80 | |
| 297 g | Methyl methacrylate | 75 |
| 99 g | Methacrylic acid | 25 |
| 2.4 g | n-Dodecyl mercaptan | 0.60 |

### Preparation Feed II

The total amount of the 15% strength by weight aqueous solution of sodium lauryl sulfate was added, with stirring, to the initially introduced, deionized water. The corresponding amounts of methyl methacrylate, Tween™80, methacrylic acid and n-dodecyl mercaptan were added in the order stated to the homogeneous solution, which was further stirred.

### Feed III:

| | | % by wt. of hydrogen peroxide |
|---|---|---|
| | | based on the total amount of |
| | | monomer |
| 4 g | 30% strength by weight | 0.3 |
| | solution of hydrogen peroxide | |
| | in deionized water | |

### Feed IV:

| | | % by wt., based on total amount of |
|---|---|---|
| | | monomer |
| 40 g | 10% strength by weight | 1.01 |
| | solution of ammonium | |
| | hydrogen carbonate in | |
| | deionized water | |

### Preparation Comparative Example 3 (C3)

The following feeds were prepared with stirring at 20° C.

Feed 1

| | |
|---|---|
| 207 g | MMA |
| 69 g | MAA |
| 5.5 g | Laromer ® BDDA |
| 2 g | Mercaptoethanol |
| 200 g | Ethanol |

Feed 2

| | |
|---|---|
| 7 g | Wako ® V 59 |
| 50 g | Ethanol |

At 20° C., a mixture of 300 g of ethanol, 15% by weight of the total amount of feed 1 and 15% by weight of the total amount of feed 2 was prepared. The mixture was heated to 78° C. under atmospheric pressure. While retaining the polymerization temperature, after 78° C. had been reached, feed 1 and feed 2 were started. Feed 1 was metered in over the course of 3 h, and feed 2 was metered in over the course of 4 h with constant feed stream. When feed 2 was complete, the reaction mixture was held for a further 2 h at 78° C. and then cooled to room temperature (ca. 20° C.).

### Preparation Example 1

The following feeds were prepared with stirring at 20° C.:

Feed 1

| | |
|---|---|
| 111 g | MMA |
| 22.5 g | MAA |
| 15 g | AA |
| 100 g | Ethanol |

Feed 2

| | |
|---|---|
| 1.5 g | Wako ® V 59 |
| 50 g | Ethanol |

At 20° C., a mixture of 150 g of ethanol, 15% by weight of the total amount of feed 1, and 15% by weight of the total amount of feed 2 was prepared. The mixture was heated to 78° C. under atmospheric pressure. While retaining the polymerization temperature, after 78° C. had been reached, feed 1 and feed 2 were started. Feed 1 was metered in over the course of 3 h, and feed 2 was metered in over the course of 4 h with constant feed stream. When feed 2 was complete, the reaction mixture was kept at 78° C. for a further 2 h and then cooled to room temperature (ca. 20° C.).

### Preparation Example 2

The following feeds were prepared with stirring at 20° C.:

Feed 1

| | |
|---|---|
| 114 g | MMA |
| 23 g | MAA |
| 15.4 g | AA |
| 1.54 g | Laromer ® TPGDA |
| 100 g | Ethanol |

Feed 2

| | |
|---|---|
| 2.25 g | Wako ® V 59 |
| 50 g | Ethanol |

At 20° C., a mixture of 150 g of ethanol, 15% by weight of the total amount of feed 1, and 15% by weight of the total amount of feed 2 was prepared. The mixture was heated to 78° C. under atmospheric pressure. While retaining the polymerization temperature, after 78° C. had been reached, feed 1 and feed 2 were started. Feed 1 was metered in over the course of 3 h, and feed 2 was metered in over the course of 4 h with constant feed stream. When feed 2 was complete, the reaction mixture was kept for a further 2 h at 78° C. and then cooled to room temperature (ca. 20° C.).

### Preparation Example 3

The following feeds were prepared with stirring at 20° C.:

Feed 1

| | |
|---|---|
| 120 g | MMA |
| 15 g | MAA |
| 15 g | AA |
| 175 g | Ethanol |

Feed 2

| | |
|---|---|
| 3 g | Sodium peroxodisulfate |
| 75 g | Water |

At 20° C., a mixture of 100 g of ethanol, 15% by weight of the total amount of feed 1, and 15% by weight of the total amount of feed 2 was prepared. The mixture was heated to 78° C. under atmospheric pressure. While retaining the polymerization temperature, after 78° C. had been reached, feed 1 and feed 2 were started. Feed 1 was metered in over the course of 3 h, and feed 2 was metered in over the course of 4 h with constant feed stream. When feed 2 was complete, the reaction mixture was kept for a further 2 h at 78° C. and then cooled to room temperature (ca. 20° C.).

### Preparation Example 4

In a 2 l polymerization vessel with stirrer and heating and cooling devices were introduced, at a temperature of from 20 to 25° C.,

| | |
|---|---|
| 350 g | of deionized water |
| 0.8 g | of a 15% strength by weight aqueous solution of sodium |
| | lauryl sulfate in deionized water |
| 35 g | of feed II (see below) |

and heated to 45° C. with stirring and under a nitrogen atmosphere. After reaching this temperature, feed I was added over the course of 5 minutes. The mixture was then heated to 80° C. and, while stirring and retaining the reaction temperature, feed II was metered in over the course of 2.5 hours with constant feed streams. When the feeds were complete, the reaction mixture was stirred for a further hour at 80° C. and then cooled to 60°. While retaining the temperature of 60°, feed III was added. The mixture was then cooled to 35° C. and, while retaining the reaction temperature, feed IV was added.

### Feed I:

| | |
|---|---|
| 5.5 g | 7% strength by weight aqueous solution of sodium persulfate in |
| | deionized water |

Feed II is an Aqueous Monomer Emulsion Prepared from:

| | | % by wt. based on the |
|---|---|---|
| Initial weight | | total amount of monomer |
| 140 g | deionized water | |
| 8 g | a 15% strength by weight | |
| | solution of sodium lauryl | |
| | sulfate in deionized water | |
| 10 g | Tween ™ 80 | |
| 250 g | Methyl methacrylate | 80 |
| 30 g | Methacrylic acid | 10 |
| 30 g | Acrylic acid | 10 |
| 2.4 g | n-Dodecyl mercaptan | 0.76 |

### Preparation Feed II

With stirring, the total amount of the 15% strength by weight aqueous solution of sodium lauryl sulfate is added to the initially introduced deionized water. The corresponding amounts of methyl methacrylate, Tween™ 80, methacrylic acid, acrylic acid and n-dodecyl mercaptan were added in the order stated to the homogeneous solution, which was further stirred.

### Feed III:

| | | % by wt. of hydrogen peroxide |
|---|---|---|
| | | based on the total amount of |
| | | monomer |
| 2 g | 30% strength by weight | 0.3 |
| | solution of hydrogen peroxide | |
| | in deionized water | |

### Feed IV:

| | | % by wt. based on the total |
|---|---|---|
| | | amount of monomer |
| 30 g | 10% strength by weight | 1.01 |
| | solution of ammonium | |
| | hydrogen carbonate in | |
| | deionized water | |

### Preparation Example 5

In a 2 l polymerization vessel with stirrer and heating and cooling devices were introduced, at a temperature of from 20 to 25° C.,

| | |
|---|---|
| 350 g | of deionized water |
| 0.8 g | of a 15% strength by weight aqueous solution of sodium lauryl |
| | sulfate in deionized water |
| 35 g | of feed II (see below) |

and heated to 45° C. with stirring and under a nitrogen atmosphere. After this temperature had been reached, feed I was added over the course of 5 minutes. The mixture was then heated to 80° C. and, while stirring and retaining the reaction temperature, feed II was added over the course of 2.5 hours with constant feed streams. When the feeds were complete, the reaction mixture was stirred for a further hour at 80° C. and then cooled to 60°. While retaining the temperature of 60°, feed III was added. The mixture was then cooled to 35° C. and, while retaining the reaction temperature, feed IV was added.

### Feed I:

| | |
|---|---|
| 5.5 g | 7% strength by weight aqueous solution of sodium persulfate in |
| | deionized water |

Feed II was an Aqueous Monomer Emulsion Prepared from:

| | | % by wt. based on the |
|---|---|---|
| Initial weight | | total amount of monomer |
| 140 g | deionized water | |
| 8 g | a 15% strength by weight | |
| | solution of sodium lauryl | |
| | sulfate in deionized water | |
| 10 g | Tween ™ 80 | |
| 250 g | Methyl methacrylate | 80 |
| 30 g | Methacrylic acid | 10 |
| 30 g | Acrylic acid | 10 |
| 6.3 g | Laromer ® BDDA | 2 |
| 2.4 g | n-Dodecyl mercaptan | 0.78 |

### Preparation Feed II

The total amount of the 15% strength by weight aqueous solution of sodium lauryl sulfate was added with stirring to the initially introduced deionized water. The corresponding amounts of methyl methacrylate, Laromer®BDDA, Tween™ 80, methacrylic acid, acrylic acid and n-dodecyl mercaptan were added in the order stated to the homogeneous solution, which was further stirred.

### Feed III:

| | | % by wt. of hydrogen |
|---|---|---|
| | | peroxide based on the |
| | | total amount of monomer |
| 2 g | 30% strength by weight | 0.3 |
| | solution of hydrogen | |
| | peroxide in deionized water | |

### Feed IV:

| | | % by wt. based on the |
|---|---|---|
| | | total amount of monomer |
| 30 g | 10% strength by weight | 1.01 |
| | solution of ammonium | |
| | hydrogen carbonate in | |
| | deionized water | |

### Preparation Example 6

The following feeds were prepared with stirring at 20° C.:

Feed 1

| | |
|---|---|
| 225 g | MMA |
| 60 g | MAA |
| 15 g | AA |
| 350 g | Ethanol |

Feed 2

| | |
|---|---|
| 7.5 g | Wako ® V 50 |
| 150 g | dem. water |

At 20° C., a mixture of 200 g of ethanol, 15% by weight of the total amount of feed 1, and 15% by weight of the total amount of feed 2 was prepared. The mixture was heated to 78° C. under atmospheric pressure. While retaining the polymerization temperature, after 78° C. had been reached, feeds 1 and 2 were started. Feed 1 was metered in over the course of 3 hours, and feed 2 was metered in over the course of 4 hours with constant feed stream. When feed 2 was complete, the reaction mixture was kept at 78° C. for a further 2 h and then cooled to room temperature (ca. 20° C.).

### Preparation Example 7

The following feeds were prepared with stirring at 20° C.:

Feed 1

| | | |
|---|---|---|
| 240 | g | MMA |
| 30 | g | MAA |
| 30 | g | AA |
| 350 | g | Ethanol |

Feed 2

| | | |
|---|---|---|
| 4.5 | g | Wako ® V 50 |
| 150 | g | dem. water |

At 20° C., a mixture of 200 g of ethanol, 15% by weight of the total amount of feed 1, and 15% by weight of the total amount of feed 2 was prepared. The mixture was heated to 78° C. under atmospheric pressure. While retaining the polymerization temperature, after 78° C. had been reached, feeds 1 and 2 were started. Feed 1 was metered in over the course of 3 hours, and feed 2 was metered in over the course of 4 hours with constant feed stream. When feed 2 was complete, the reaction mixture was held at 78° C. for a further 2 h and then cooled to room temperature (ca. 20° C.).

### Application Properties

| | | | Ability to be | PSD-Malvern | Curl | |
|---|---|---|---|---|---|---|
| | Composition | Setting* | washed out | [μm]** | retention [%] | Stickiness |
| C1 | MMA/AA | 75 | good | 50 | 40 | 3 |
| | 75/25 | | | | | |
| C2 | MMA/MAA | 92 | poor | 73 | 83 | 0 |
| | 75/25 | | | | | |
| C3 | MMA/MAA/ | — | poor, | 32 | — | — |
| | Laromer ® BDDA | | stuck | | | |
| | 73/25/2 | | | | | |
| 1 | MMA/MAA/AA | 108 | just good | 40 | 76 | 0 |
| | 75/15/10 | | | | | |
| 2 | MMA/MAA/AA | 110 | just good- | 42 | — | 1 |
| | Laromer ® TPGDA | | good | | | |
| | 74/15/10/1 | | | | | |
| 4 | MMA/MAA/AA | 110 | good | 65 | 92 | 0 |
| | 80/10/10 | | | | | |
| 6 | MMA/MAA/AA | 120 | good | 35 | 70 | 0 |
| | 75/20/5 | | | | | |
| 7 | MMA/MAA/AA | 120 | good | 35 | 84 | 0 |
| | 80/10/10 | | | | | |

*Determination of the setting relative [%] to the standard Amphomer ®LV71
**VOC55 Aerosol:
5% of the particular polymer, completely neutralized with AMP,
40% DME,
15% ethanol,
40% water;
Spray device:
Spray head: Kosmos .020D Wirbel .018″ 21-6443-20 (Precision Valve),
Valve: DPV 33876 (Precision Valve)

### II) Application Examples

Unless stated otherwise, all of the polymers containing acid groups used are 100% neutralized with AMP. “Water ad 100” means that the residual amount of water necessary to achieve a total amount of 100% by weight is added to the particular preparation.

Unless determined otherwise, the % quantities are % by weight.

The abbreviation “q.s.” means “quantum satis”, i.e. sufficient of an ingredient is added as is necessary to achieve a desired effect.

The designation (solid) means that the amount of the polymer used is calculated on the basis of the solids fraction if the polymer is present in solution.

### Example 1a

### VOC 55 Aerosol Hair Spray

| | [%] |
|---|---|
| Polymer from example No. 1 (solid) | 5.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1b

### VOC 55 Aerosol Hair Spray

| | [%] |
|---|---|
| Polymer from example No. 1 (solid) | 3.00 |
| Balance ® 0/55 (National Starch) | 2.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1c

### VOC 55 Aerosol Hair Spray

| | [%] |
|---|---|
| Polymer from example No. 1 (solid) | 3.00 |
| Acudyne ® 180 (Rohm & Haas) | 1.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1d

### VOC 55 Aerosol Hair Spray

| | [%] |
|---|---|
| Polymer from example No. 1 (solid) | 3.00 |
| Amphomer ® LV 71 (National Starch) | 2.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1e

### VOC 55 Aerosol Hair Spray

| | [%] |
|---|---|
| Polymer from example No. 1 (solid) | 3.00 |
| Acudyne ® DHR (Rohm & Haas) | 1.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1e

### VOC 55 Aerosol Hair Spray

| | [%] |
|---|---|
| Polymer from example No. 1 (solid) | 3.00 |
| Eastman ® AQ 48 (Eastman-Kodak) | 2.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1f

### VOC 55 Aerosol Hair Spray

| | [%] |
|---|---|
| Polymer from example No. 1 (solid) | 3.00 |
| Resyn ® 28-2930 (National Starch) | 2.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1g

### VOC 55 Aerosol Hair Spray

| | [%] |
|---|---|
| Polymer from example No. 1 (solid) | 3.00 |
| Balance ® 47 (National Starch) | 2.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 1h

### VOC 55 Aerosol Hair Spray

| | [%] |
|---|---|
| Polymer from example No. 1 (solid) | 3.00 |
| Aquaflex ® SF-40 (ISP) | 1.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 11

### VOC 55 Aerosol Hair Spray

| | [%] |
|---|---|
| Polymer from example No. 1 (solid) | 3.00 |
| DynamX ® (ISP) | 1.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2a

### VOC 55 Aerosol Hair Spray

| | [%] |
|---|---|
| Polymer from example No. 1 (solid) | 5.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2b

### VOC 55 Aerosol Hair Spray

| | [%] |
|---|---|
| Polymer from example No. 1 (solid) | 3.00 |
| Balance ® 0/55 (National Starch) | 2.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2c

### VOC 55 Aerosol Hair Spray

| | |
|---|---|
| Polymer from example No. 1 (solid) | 3.00 |
| Acudyne ® 180 © Rohm & Haas) | 1.00 |
| Dimethyl ether | 40.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2d

### VOC 55 Aerosol Hair Spray

| | [%] |
|---|---|
| Polymer from example No. 1 (solid) | 3.00 |
| Amphomer ® LV 71 (National Starch) | 2.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2e

### VOC 55 Aerosol Hair Spray

| | [%] |
|---|---|
| Polymer from example No. 1 (solid) | 3.00 |
| Acudyne ® DHR (Rohm & Haas) | 1.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2e

### VOC 55 Aerosol Hair Spray

| | [%] |
|---|---|
| Polymer from example No. 1 (solid) | 3.00 |
| Eastman ® AQ 48 (Eastman-Kodak) | 2.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2f

### VOC 55 Aerosol Hair Spray

| | [%] |
|---|---|
| Polymer from example No. 1 (solid) | 3.00 |
| Resyn ® 28-2930 (National Starch) | 2.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2g

### VOC 55 Aerosol Hair Spray

| | [%] |
|---|---|
| Polymer from example No. 1 (solid) | 3.00 |
| Balance ® 47 (National Starch) | 2.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2h

### VOC 55-Aerosol Hair Spray

| | [%] |
|---|---|
| Polymer from example No. 1 (solid) | 3.00 |
| Aquaflex ® SF-40 (ISP) | 1.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 2i

### VOC 55 Aerosol Hair Spray

| | [%] |
|---|---|
| Polymer from example No. 1 (solid) | 3.00 |
| DynamX ® (ISP) | 1.00 |
| Dimethyl ether | 35.00 |
| Propane/butane | 5.00 |
| Ethanol | 15.00 |
| Water | ad 100 |

further additives: silicone, perfume, antifoam, UV absorber

The example can be repeated with each of the polymers 2-7 according to the invention. In each case, a VOC 55 aerosol hair spray with good properties is obtained.

### Example 3a

### Aerosol Hair Spray with Fluorocarbon Propellants

| | [%] |
|---|---|
| Polymer from example No. 1 (solid) | 5.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3b

### VOC 55 Aerosol Hair Spray

| | [%] |
|---|---|
| Polymer from example No. 1 (solid) | 3.00 |
| Balance ® 0/55 (National Starch) | 2.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3d

### VOC 55 Aerosol Hair Spray

| | [%] |
|---|---|
| Polymer from example No. 1 (solid) | 3.00 |
| Acudyne ® 180 (Rohm & Haas) | 1.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3d

### VOC 55 Aerosol Hair Spray

| | [%] |
|---|---|
| Polymer from example No. 1 (solid) | 3.00 |
| Amphomer ® LV 71 (National Starch) | 2.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3e

### VOC 55 Aerosol Hair Spray

| | [%] |
|---|---|
| Polymer from example No. 1 (solid) | 3.00 |
| Acudyne ® DHR (Rohm & Haas) | 1.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3e

### VOC 55 Aerosol Hair Spray

| | [%] |
|---|---|
| Polymer from example No. 1 (solid) | 3.00 |
| Eastman ® AQ 48 (Eastman-Kodak) | 2.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3f

### VOC 55 Aerosol Hair Spray

| | [%] |
|---|---|
| Polymer from example No. 1 (solid) | 3.00 |
| Resyn ® 28-2930 (National Starch) | 2.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3g

### VOC 55 Aerosol Hair Spray

| | [%] |
|---|---|
| Polymer from example No. 1 (solid) | 3.00 |
| Balances ® 47 (National Starch) | 2.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3h

### VOC 55 Aerosol Hair Spray

| | [%] |
|---|---|
| Polymer from example No. 1 (solid) | 3.00 |
| Aquaflex ® SF-40 (ISP) | 1.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 3i

### VOC 55 Aerosol Hair Spray

| | [%] |
|---|---|
| Polymer from example No. 1 (solid) | 3.00 |
| DynamX ® (ISP) | 1.00 |
| Ethanol abs. | ad 100 |
| HFC 152A | 40.00 |

further additives: silicone, perfume, antifoam, UV absorber

The example can be repeated with each of the polymers 2-7 according to the invention. In each case, an aerosol hair spray with good properties is obtained.

### Example 4

### Aerosol Hair Spray with Fluorocarbon Propellants

| | [%] |
|---|---|
| Polymer from example No. 1 (solid) | 5.00 |
| Dist. water | ad 100 |
| HFC 152A | 10.00 |
| Dimethyl ether | 30.00 |
| Ethanol abs. | 30.00 |

further additives: silicone, perfume, antifoam, UV absorber

The example can be repeated with each of the polymers 1-11 and 13-15. In each case, an aerosol hair spray with good properties is obtained.

### Example 5

### VOC 55 Aerosol Hair Spray

| | [%] |
|---|---|
| Polymer from example No. 1 (solid) | 3.00 |
| Ultrahold ® Strong (solid, BASF) | 1.00 |
| Dimethyl ether | 40.00 |
| Ethanol | 15.00 |
| +AMP | to pH 8.3 |
| Water | ad 100 |

further additive: silicone, perfume, antifoam

The example can be repeated with each of the polymers 2-7 according to the invention. In each case, a VOC 55 aerosol hair spray with good properties is obtained.

### Example 6

### VOC 55 Aerosol Hair Spray

| | [%] |
|---|---|
| Polymer from example No. 1 (solid) | 3.00 |
| Luvimer ® Pro55 (solid, BASF) | 1.00 |
| Dimethyl ether | 40.00 |
| Ethanol | 15.00 |
| Water | ad 100 |

further additive: silicone, perfume, antifoam

The example can be repeated with each of the polymers 2-7 according to the invention. In each case, a VOC 55 aerosol hair spray with good properties is obtained.

### Example 7

### VOC 55 Aerosol Hair Spray

| | [%] |
|---|---|
| Polymer from example No. 1 (solid) | 3.00 |
| Luvimer ® P.U.R (solid, BASF) | 1.00 |
| Dimethyl ether | 40.00 |
| Ethanol | 15.00 |
| Water | ad 100 |

further additive: silicone, perfume, antifoam

The example can be repeated with each of the polymers 2-7 according to the invention. In each case, a VOC 55 aerosol hair spray with good properties is obtained.

### Example 8

### VOC 55 Aerosol Hair Spray

| | [%] |
|---|---|
| Polymer from example No. 1 (solid) | 3.00 |
| Resyn ® 28-2930 (solid, National Starch) | 1.00 |
| Dimethyl ether | 40.00 |
| Ethanol | 15.00 |
| Water | ad 100 |

further additive: silicone, perfume, antifoam

The example can be repeated with each of the polymers 2-7 according to the invention. A VOC 55 aerosol hair spray with good properties is likewise obtained.

### Example 9

### VOC 55 Aerosol Hair Spray

| | [%] |
|---|---|
| Polymer from example No. 1 (solid) | 2.00 |
| Stepanhold ® R-1*⁾ (Stepan Chemical Co.) | 1.00 |
| Dimethyl ether | 40.00 |
| Ethanol | 15.00 |
| +AMP | to pH 8.3 |
| Water | ad 100 |

further additive: silicone, perfume, antifoam
*⁾Stepanhold ® R-1 = poly(vinylpyrrolidone/ethyl methacrylate/methacrylic acid)

The example can be repeated with each of the polymers 2-7 according to the invention. In each case, a VOC 55 aerosol hair spray with good properties is obtained.

### Example 10

### VOC 55 Handpump Spray

| | [%] |
|---|---|
| Polymer from example No. 1 (solid) | 7.00 |
| Ethanol | 55.00 |
| Water | ad 100 |

further additive: silicone, perfume, antifoam

The example can be repeated with each of the polymers 2-7 according to the invention. In each case, a VOC 55 handpump spray with good properties is obtained.

### Example 11

### VOC 80 Aerosol Hair Spray

| | [%] |
|---|---|
| Polymer from example No. 1 (solid) | 12.00 |
| Dimethyl ether | 40.00 |
| Ethanol | 40.00 |
| Water | ad 100 |

further additive: silicone, perfume, antifoam

The example can be repeated with each of the polymers 2-7 according to the invention. In each case, a VOC 80 aerosol hair spray with good properties is obtained.

### Example 11

| Aqueous handpump spray | [%] |
|---|---|
| Polymer from example No. 1 (solid) | 4.00 |
| Luviset ® Clear*⁾ (solid) | 1.00 |
| Water | ad 100 |

further additive, water-soluble silicone, perfume, antifoam.
*⁾Luviset ® Clear: poly(vinylpyrrolidone/methacrylamide/vinylimidazole), BASF

The example can be repeated with each of the polymers 2-7 according to the invention. In each case, an aqueous handpump spray with good properties is obtained.

### Example 12

| Aqueous/ethanolic setting solution | [%] |
|---|---|
| Polymer from example No. 1 (solid) | 7.00 |
| Dist. water | ad 100 |
| Ethanol | 52.00 |

further additive: silicone, perfume, antifoam

The example can be repeated with each of the polymers 2-7 according to the invention. In each case, a setting solution with good properties is obtained.

### Example 13

| Ethanolic setting solution | [%] |
|---|---|
| Polymer from example No. 1 (solid) | 7.0 |
| Ethanol | ad 100 |

further additive: silicone, perfume, antifoam . . .

The example can be repeated with each of the polymers 2-7 according to the invention. In each case, a setting solution with good properties is obtained.

### Example 14

| Hair gel with Aculyn 28: | [%] |
|---|---|
| Phase 1: | |
| Polymer from example No. 1 (solid) | 6.00 |
| Aminomethylpropanol (38% strength solution) | 1.0 |
| Water, dist. | ad 50 |

further additive: preservative, soluble ethoxylated silicone, perfume . . .

| | |
|---|---|
| Phase 2: | |
| Aculyn 28 (1% strength aqueous suspension) | 50.00 |

Preparation:
Phases 1 and 2 are weighed in separately and homogenized. Phase 2 is then slowly stirred into phase 1. An essentially clear, stable gel is formed.

The example can be repeated with each of the polymers 2-7 according to the invention. In each case, a hair gel containing Aculyn 28 with good properties is obtained.

### Example 15

| Hair gel with hydroxyethylcellulose: | [%] |
|---|---|
| Phase 1: | |
| Polymer from example No. 1 (solid) | 6.00 |
| Water, dist. | ad 50 |

further additive: preservative, soluble ethoxylated silicone, perfume

| | |
|---|---|
| Phase 2: | |
| Natrosol HR 250 (5% strength solution) | 50.00 |
| Hydroxyethylcellulose (Hercules). | |

Preparation:
Phases 1 and 2 are weighed in separately and homogenized. Phase 2 is then slowly stirred into phase 1. An essentially clear, stable gel is formed.

The example can be repeated with each of the polymers 2-7 according to the invention. In each case, a hair gel containing hydroxyethylcellulose with good properties is obtained.

### Example 16

| Foam conditioner | [%] |
|---|---|
| Polymer from example No. 1 (solid) | 0.50 |
| Cremophor ® A 25 (Ceteareth 25/BASF) | 0.20 |
| Comperlan ® KD (Coamide DEA/Henkel) | 0.10 |
| Propane/butane | 10.00 |

further additive: perfume, preservative

| | |
|---|---|
| Water | ad 100 |

Preparation:
Weigh in and dissolve with stirring. Bottle and add propellant gas.

The example can be repeated with each of the polymers 2-7 according to the invention. In each case, a foam conditioner with good properties is obtained.

### Example 17

| Conditioner shampoo: | | [%] |
|---|---|---|
| A) | Texapon ® NSO 28% strength (sodium laureth sulfate/ | 50.00 |
| | Henkel) | |
| | Comperlan ® KS (Coamide DEA/Henkel) | 1.00 |
| | Polymer from example No. 1 (solid) | 3.00 |
| | q.s. perfume oil | |
| B) | Water | 44.5 |
| | Sodium chloride | 1.5 |
| | q.s. preservative | |

Preparation:
Phases 1 and 2 are weighed in separately and homogenized. Phase 2 is then slowly stirred into phase 1. An essentially clear, stable gel is formed.

The example can be repeated with each of the polymers 2-7 according to the invention. In each case, a conditioner shampoo with good properties is obtained.

### Example 18

Standard O/W cream:

| | [%] | CTFA name |
|---|---|---|
| Oil phase: | | |
| Cremophor ® A6 | 3.5 | Ceteareth-6 (and) Stearyl |
| | | Alcohol |
| Cremophor ® A25 | 3.5 | Ceteareth-25 |
| Glycerol monostearate s.e. | 2.5 | Glyceryl stearate |
| Paraffin oil | 7.5 | Paraffin Oil |
| Cetyl alcohol | 2.5 | Cetyl Alcohol |
| Luvitol ® EHO | 3.2 | Cetearyl Octanoate |
| Vitamin E acetate | 1.0 | Tocopheryl Acetate |
| Nip-Nip | 0.1 | Methyl and propyl 4- |
| | | hydroxybenzoate |
| | | (7:3) |
| Water phase: | | |
| Polymer from example No. 1 (solid) | 0.6 | |
| Water | 77.0 | |
| 1,2-Propylene glycol | 1.5 | Propylene glycol |
| Germall II | 0.1 | Imidazolidinylurea |

Preparation:
The oil and water phases are weighed in separately and homogenized at a temperature of about 80° C. The water phase is then slowly stirred into the oil phase and cooled slowly to room temperature with stirring.

The example can be repeated with each of the polymers 2-7 according to the invention. In each case, a standard O/W cream with good properties is obtained.

### Example 19

### Liquid Makeup

A

| | |
|---|---|
| 1.70 | Glyceryl stearate |
| 1.70 | Cetyl alcohol |
| 1.70 | Ceteareth-6 |
| 1.70 | Ceteareth-25 |
| 5.20 | Caprylic/capric triglyceride |
| 5.20 | Mineral oil |

B

| | |
|---|---|
| q.s. | Preservative |
| 4.30 | Propylene glycol |
| 2.50 | Polymer from example 1 (solid) |
| ad 100 | Dist. water |

C

| | |
|---|---|
| q.s. | Perfume oil |

D

| | |
|---|---|
| 2.00 | Iron oxide |
| 12.00 | Titanium dioxide |

Preparation:
Heat phase A and phase B separately from one another to 80° C. Then mix phase B into phase A using a stirrer. Allow everything to cool to 40° C. and add phase C and phase D. Homogenize again.

The example can be repeated with each of the polymers 2-7 according to the invention. In each case, a liquid makeup with good properties is obtained.

### Example 20

### Oil-Free Makeup

A

| | |
|---|---|
| 0.35 | Veegum |
| 5.00 | Butylene glycol |
| 0.15 | Xanthan gum |

B

| | |
|---|---|
| 34.0 | Dist. water |
| q.s. | Preservative |
| 0.2 | Polysorbate-20 |
| 1.6 | Tetrahydroxypropylethylenediamine |

C

| | |
|---|---|
| 1.0 | Silicon dioxide |
| 2.0 | Nylon-12 |
| 4.15 | Mica |
| 6.0 | Titanium dioxide |
| 1.85 | Iron oxide |

D

| | |
|---|---|
| 4.0 | Stearic acid |
| 1.5 | Glyceryl stearate |
| 7.0 | Benzyl laurate |
| 5.0 | Isoeicosane |
| q.s. | Preservative |

E

| | |
|---|---|
| 0.5 | Panthenol |
| 0.1 | Imidazolidinylurea |
| 5.0 | Polymer from example 1 (solid) |

Preparation:
Wet phase A with butylene glycol, add to phase B and mix well. Heat phase AB to 75° C. Pulverize phase C feed materials, add to phase AB and homogenize well. Mix feed materials of phase D, heat to 80° C. and add to phase ABC. Mix for some time until everything is homogeneous. Transfer everything to a vessel with propeller mixer. Mix feed materials of phase E, add to phase ABCD and mix well.

The example can be repeated with each of the polymers 2-7 according to the invention. In each case, an oil-free makeup with good properties is obtained.

### Example 21

### Shimmering Gel

A

| | |
|---|---|
| 32.6 | Dist. water |
| 0.1 | Disodium-EDTA |
| 25.0 | Natrosol (4% strength aqueous solution) |
| 0.3 | Preservative |

B

| | |
|---|---|
| 0.5 | Dist. water |
| 0.5 | Triethanolamine |

C

| | |
|---|---|
| 2.0 | Polymer from example 1 (solid) |
| ad 100 | Dist. water |
| 1.0 | Polyquaternium-46 (20% strength aqueous solution) |
| 5.0 | Iron oxide |

D

| | |
|---|---|
| 15.0 | Dist. water |
| 1.0 | D-Panthenol 50 P (Panthenol and propylene glycol) |

Preparation:
Using a propeller mixer, thoroughly mix the feed materials of phase A in the order stated. Then add phase B to phase A. Stir slowly until everything is homogeneous. Thoroughly homogenize phase C until the pigments are well distributed. Add phase C and phase D to phase AB and mix well.

The example can be repeated with each of the polymers 2-7 according to the invention. In each case, a shimmering gel with good properties is obtained.

### Example 22

### Sunscreen Gel

Phase A

| | |
|---|---|
| 1.00 | Hydrogenated castor oil PEG-40 |
| 8.00 | Octyl methoxycinnamate (Uvinul ® MC 80) |
| 5.00 | Octocrylene (Uvinul ® N 539) |
| 0.80 | Octyl triazone (Uvinul ® T 150) |
| 2.00 | Butyl methoxydibenzoylmethane (Uvinul ® BMBM) |
| 2.00 | Tocopheryl acetate |
| q.s. | Perfume oil |

Phase B

| | |
|---|---|
| 2.50 | Polymer from example 1 (solid) |
| ad 100 | Dist. water |
| 0.30 | Acrylate/C₁₀₋₃₀ alkyl acrylate copolymer |
| 0.20 | Carbomer |
| 5.00 | Glycerol |
| 0.20 | Disodium EDTA |
| q.s. | Preservative |
| 62.80 | Dist. water |

Phase C

| | |
|---|---|
| 0.20 | Sodium hydroxide |

Preparation:
Mix the components of phase A. Allow phase B to swell and stir into phase A with homogenization. Neutralize with phase C and homogenize again.

The example can be repeated with each of the polymers 2-7 according to the invention. In each case, a sunscreen gel with good properties is obtained.

### Example 23

### Sunscreen emulsion containing TiO₂ and ZnO₂

Phase A

| | |
|---|---|
| 6.00 | Hydrogenated castor oil PEG-7 |
| 2.00 | PEG-45/dodecyl glycol copolymer |
| 3.00 | Isopropyl myristate |
| 8.00 | Jojoba oil (Buxus Chinensis) |
| 4.00 | Octyl methoxycinnamate (Uvinul ® MC 80) |
| 2.00 | 4-Methylbenzylidene camphor (Uvinul ® MBC 95) |
| 3.00 | Titanium dioxide, dimethicone |
| 1.00 | Dimethicone |
| 5.00 | Zinc oxide, dimethicone |

Phase B

| | |
|---|---|
| 2.0 | Polymer from example 1 (solid) |
| ad 100 | Dist. water |
| 0.20 | Disodium EDTA |
| 5.00 | Glycerol |
| q.s. | Preservative |
| 50.80 | Dist. water |

Phase C

| | |
|---|---|
| q.s. | Perfume oil |

Preparation:
Heat phases A and B separately to about 85° C. Stir phase B into phase A and homogenize. Cool to about 40° C., add phase C and briefly homogenize again.

The example can be repeated with each of the polymers 2-7 according to the invention. In each case, a sunscreen emulsion containing TiO₂ and ZnO₂ with good properties is obtained.

### Example 24

### Sunscreen Lotion

Phase A

| | |
|---|---|
| 6.00 | Octyl methoxycinnamate (Uvinul ® MC 80) |
| 2.50 | 4-Methylbenzylidene camphor (Uvinul ® MBC 95) |
| 1.00 | Octyl triazone (Uvinul ® T 150) |
| 2.00 | Butyl methoxydibenzoylmethane (Uvinul ® BMBM) |
| 2.00 | PVP/hexadecene copolymer |
| 5.00 | PPG-3 myristyl ether |
| 0.50 | Dimethicone |
| 0.10 | BHT, ascorbyl palmitate, citric acid, glyceryl stearate, propylene |
| | glycol |
| 2.00 | Cetyl alcohol |
| 2.00 | Potassium cetyl phosphate |

Phase B

| | |
|---|---|
| 0.50 | Polymer from example 1 (solid) |
| ad 100 | Dist. water |
| 5.00 | Propylene glycol |
| 0.20 | Disodium EDTA |
| q.s. | Preservative |
| 63.92 | Dist. water |

Phase C

| | |
|---|---|
| 5.00 | Mineral oil |
| 0.20 | Carbomer |

Phase D

| | |
|---|---|
| 0.08 | Sodium hydroxide |

Phase E

| | |
|---|---|
| q.s. | Perfume oil |

Preparation:
Heat phases A and B separately to about 80° C. Stir phase B into phase A with homogenization, briefly afterhomogenize. Slurry phase C, stir into phase AB, neutralize with phase D and afterhomogenize. Cool to about 40° C., add phase E, homogenize again.

The example can be repeated with each of the polymers 2-7 according to the invention. In each case, a sunscreen lotion with good properties is obtained.

### Example 25

### Peelable Face Mask

Phase A

| | |
|---|---|
| 57.10 | Dist. water |
| 6.00 | Polyvinyl alcohol |
| 5.00 | Propylene glycol |

Phase B

| | |
|---|---|
| 20.00 | Alcohol |
| 4.00 | PEG-32 |
| q.s | Perfume oil |

Phase C

| | |
|---|---|
| 5.00 | Polyquaternium-44 |
| 0.50 | Polymer from example 1 (solid) |
| ad 100 | Dist. water |
| 0.20 | Allantoin |

Preparation:
Heat phase A to at least 90° C. and stir until dissolved. Dissolve phase B at 50° C. and stir into phase A. At about 35° C., make up the loss of ethanol. Add phase C and stir in.

The example can be repeated with each of the polymers 2-7 according to the invention. In each case, a peelable face mask with good properties is obtained.

### Example 26

### Face Mask

Phase A

| | |
|---|---|
| 3.00 | Ceteareth-6 |
| 1.50 | Ceteareth-25 |
| 5.00 | Cetearyl alcohol |
| 6.00 | Cetearyl octanoate |
| 6.00 | Mineral oil |
| 0.20 | Bisabolol |
| 3.00 | Glyceryl stearate |

Phase B

| | |
|---|---|
| 2.00 | Propylene glycol |
| 5.00 | Panthenol |
| 2.50 | Polymer from example 1 (solid) |
| ad 100 | Dist. water |
| q.s. | Preservative |
| 53.80 | Dist. water |

Phase C

| | |
|---|---|
| q.s. | Perfume oil |
| 0.50 | Tocopheryl acetate |

Preparation:
Heat phase A and B separately to about 80° C. Stir phase B into phase A with homogenization, briefly afterhomogenize. Cool to about 40° C., add phase C, homogenize again.

The example can be repeated with each of the polymers 2-7 according to the invention. In each case, a face mask with good properties is obtained.

### Example 27

### Body Lotion Foam

Phase A

| | |
|---|---|
| 1.50 | Ceteareth-25 |
| 1.50 | Ceteareth-6 |
| 4.00 | Cetearyl alcohol |
| 10.00 | Cetearyl octanoate |
| 1.00 | Dimethicone |

Phase B

| | |
|---|---|
| 0.50 | Polymer from example 1 (solid) |
| ad 100 | Dist. water |
| 2.00 | Panthenol |
| 2.50 | Propylene glycol |
| q.s. | Preservative |
| 74.50 | Dist. water |

Phase C

| | |
|---|---|
| q.s. | Perfume oil |

Preparation:
Heat phases A and B separately to about 80° C. Stir phase B into phase A and homogenize. Cool to about 40° C., add phase C and briefly homogenize again. Bottling: 90% active ingredient and 10% propane/butane at 3.5 bar (20° C.).

The example can be repeated with each of the polymers 2-7 according to the invention. In each case, a body lotion foam with good properties is obtained.

### Example 28

### Face Tonic for Dry and Sensitive Skin

Phase A

| | |
|---|---|
| 2.50 | Hydrogenated castor oil PEG-40 |
| q.s. | Perfume oil |
| 0.40 | Bisabolol |

Phase B

| | |
|---|---|
| 3.00 | Glycerol |
| 1.00 | Hydroxyethylcetyldimonium phosphate |
| 5.00 | Witchhazel (Hamamelis Virginiana) distillate |
| 0.50 | Panthenol |
| 0.1 | Polymer from example 1 (solid) |
| ad 100 | Dist. water |
| q.s. | Preservative |
| 87.60 | Dist. water |

Preparation:
Dissolve phase A to give a clear solution. Stir phase B into phase A.

The example can be repeated with each of the polymers 2-7 according to the invention. In each case, a face tonic for dry and sensitive skin with good properties is obtained.

### Example 29

### Face Washing Paste with Peeling Effect

Phase A

| | |
|---|---|
| 58.00 | Dist. water |
| 2.50 | Polymer from example 1 (solid) |
| ad 100 | Dist. water |
| 1.50 | Carbomer |
| q.s. | Preservative |

Phase B

| | |
|---|---|
| q.s. | Perfume oil |
| 7.00 | Potassium cocoyl hydrolyzed protein |
| 4.00 | Cocamidopropylbetaine |

Phase C

| | |
|---|---|
| 1.50 | Triethanolamine |

Phase D

| | |
|---|---|
| 13.00 | Polyethylene (Luwax ® A) |

Preparation:
Allow phase A to swell. Dissolve phase B to give a clear solution. Stir phase B into phase A. Neutralize with phase C. Then stir in phase D.

The example can be repeated with each of the polymers 2-7 according to the invention. In each case, a face washing paste with peeling effect with good properties is obtained.

### Example 30

### Face Soap

Phase A

| | |
|---|---|
| 25.0 | Potassium cocoate |
| 20.0 | Disodium cocoamphodiacetate |
| 2.0 | Lauramide DEA |
| 1.0 | Glycol stearate |
| 0.50 | Polymer from example 1 (solid) |
| ad 100 | Dist. water |
| 50.0 | Dist. water |
| q.s. | Citric acid |

Phase B

| | |
|---|---|
| q.s. | Preservative |
| q.s. | Perfume oil |

Preparation:
Heat phase A to 70° C. with stirring until everything is homogeneous, adjust the pH to 7.0-7.5 with citric acid, allow everything to cool to 50° C. and add phase B.

The example can be repeated with each of the polymers 2-7 according to the invention. In each case, a face soap with good properties is obtained.

### Example 31

### Face Cleansing Milk, O/W Type

Phase A

| | |
|---|---|
| 1.50 | Ceteareth-6 |
| 1.50 | Ceteareth-25 |
| 2.00 | Glyceryl stearate |
| 2.00 | Cetyl alcohol |
| 10.00 | Mineral oil |

Phase B

| | |
|---|---|
| 5.00 | Propylene glycol |
| q.s. | Preservative |
| 1.0 | Polymer from example 1 (solid) |
| ad 100 | Dist. water |
| 62.30 | Dist. water |

Phase C

| | |
|---|---|
| 0.20 | Carbomer |
| 10.00 | Cetearyl octanoate |

Phase D

| | |
|---|---|
| 0.40 | Tetrahydroxypropylethylenediamine |

Phase E

| | |
|---|---|
| q.s. | Perfume oil |
| 0.10 | Bisabolol |

Preparation:
Heat phases A and B separately to about 80° C. Stir phase B into phase A with homogenization, briefly afterhomogenize. Slurry phase C, stir into phase AB, neutralize with phase D and afterhomogenize. Cool to about 40° C., add phase E, homogenize again.

The example can be repeated with each of the polymers 2-7 according to the invention. In each case, a face cleansing milk, O/W type with good properties is obtained.

### Example 32

### Peeling Cream, O/W Type

Phase A

| | |
|---|---|
| 3.00 | Ceteareth-6 |
| 1.50 | Ceteareth-25 |
| 3.00 | Glyceryl stearate |
| 5.00 | Cetearyl alcohol, sodium cetearyl sulfate |
| 6.00 | Cetearyl octanoate |
| 6.00 | Mineral oil |
| 0.20 | Bisabolol |

Phase B

| | |
|---|---|
| 2.00 | Propylene glycol |
| 0.10 | Disodium EDTA |
| 0.50 | Polymer from example 1 (solid) |
| ad 100 | Dist. water |
| q.s. | Preservative |
| 59.70 | Dist. water |

Phase C

| | |
|---|---|
| 0.50 | Tocopheryl acetate |
| q.s. | Perfume oil |

Phase D

| | |
|---|---|
| 10.00 | Polyethylene |

Preparation:
Heat phases A and B separately to about 80° C. Stir phase B into phase A and homogenize. Cool to about 40° C., add phase C and briefly homogenize again. Then stir in phase D.

The example can be repeated with each of the polymers 2-7 according to the invention. In each case, a peeling cream, type O/W with good properties is obtained.

### Example 33

### Shaving Foam

| | |
|---|---|
| 6.00 | Ceteareth-25 |
| 5.00 | Poloxamer 407 |
| 52.00 | Dist. water |
| 1.00 | Triethanolamine |
| 5.00 | Propylene glycol |
| 1.00 | Lanolin oil PEG-75 |
| 1.0 | Polymer from example 1 (solid) |
| ad 100 | Dist. water |
| q.s. | Preservative |
| q.s. | Perfume oil |
| 25.00 | Sodium Laureth Sulfate |

Preparation:
Weigh everything together, then stir until dissolved. Bottling: 90 parts of active substance and 10 parts of propane/butane mixture 25:75.

The example can be repeated with each of the polymers 2-7 according to the invention. In each case, a shaving foam with good properties is obtained.

### Example 34

### After Shave Balm

Phase A

| | |
|---|---|
| 0.25 | Acrylate/C₁₀₋₃₀ alkyl acrylate copolymer |
| 1.50 | Tocopheryl acetate |
| 0.20 | Bisabolol |
| 10.00 | Caprylic/capric triglyceride |
| q.s. | Perfume oil |
| 1.00 | Hydrogenated castor oil PEG-40 |

Phase B

| | |
|---|---|
| 1.00 | Panthenol |
| 15.00 | Alcohol |
| 5.00 | Glycerol |
| 0.05 | Hydroxyethylcellulose |
| 0.50 | Polymer from example 1 (solid) |
| ad 100 | Dist. water |
| 64.00 | Dist. water |

Phase C

| | |
|---|---|
| 0.08 | Sodium hydroxide |

Preparation:
Mix the components of phase A. Stir phase B into phase A with homogenization, briefly after homogenize. Neutralize with phase C and homogenize again.

The example can be repeated with each of the polymers 2-7 according to the invention. In each case, an aftershave balm with good properties is obtained.

### Example 35

### Body Care Cream

Phase A

| | |
|---|---|
| 2.00 | Ceteareth-6 |
| 2.00 | Ceteareth-25 |
| 2.00 | Cetearyl alcohol |
| 3.00 | Glyceryl stearate SE |
| 5.00 | Mineral oil |
| 4.00 | Jojoba oil (Buxus Chinensis) |
| 3.00 | Cetearyl octanoate |
| 1.00 | Dimethicone |
| 3.00 | Mineral oil, lanolin alcohol |

Phase B

| | |
|---|---|
| 5.00 | Propylene glycol |
| 0.50 | Veegum |
| 1.00 | Panthenol |
| 1.70 | Polymer from example 1 (solid) |
| ad 100 | Dist. water |
| 6.00 | Polyquaternium-44 (10% strength aqueous solution) |
| q.s. | Preservative |
| 54.00 | Dist. water |

Phase C

| | |
|---|---|
| q.s. | Perfume oil |

Preparation:
Heat phases A and B separately to about 80° C. Homogenize phase B. Stir phase B into phase A with homogenization, briefly after homogenize. Cool to about 40° C., add phase C and briefly homogenize again.

The example can be repeated with each of the polymers 2-7 according to the invention. In each case, a body care cream with good properties is obtained.

### Example 36

### Toothpaste

Phase A

| | |
|---|---|
| 34.79 | Dist. water |
| 0.50 | Polymer from example 1 (solid) |
| ad 100 | Dist. water |
| 0.30 | Preservative |
| 20.00 | Glycerol |
| 0.76 | Sodium monofluorophosphate |

Phase B

| | |
|---|---|
| 1.20 | Sodium carboxymethylcellulose |

Phase C

| | |
|---|---|
| 0.80 | Aroma oil |
| 0.06 | Saccharin |
| 0.10 | Preservative |
| 0.05 | Bisabolol |
| 1.00 | Panthenol |
| 0.50 | Tocopheryl acetate |
| 2.80 | Silicon dioxide |
| 1.00 | Sodium lauryl sulfate |
| 7.90 | Dicalcium phosphate, anhydrous |
| 25.29 | Dicalcium phosphate dihydrate |
| 0.45 | Titanium dioxide |

Preparation:
Dissolve phase A. Sprinkle phase B into phase A and dissolve. Add phase C and stir under reduced pressure at RT for about 45 min.

The example can be repeated with each of the polymers 2-7 according to the invention. In each case, a toothpaste with good properties is obtained.

### Example 37

### Mouthwash

Phase A

| | |
|---|---|
| 2.00 | Aroma oil |
| 4.00 | Hydrogenated castor oil PEG-40 |
| 1.00 | Bisabolol |
| 30.00 | Alcohol |

Phase B

| | |
|---|---|
| 0.20 | Saccharin |
| 5.00 | Glycerol |
| q.s. | Preservative |
| 5.00 | Poloxamer 407 |
| 0.50 | Polymer from example 1 (solid) |
| ad 100 | Dist. water |

Preparation:
Dissolve phase A and phase B separately to give a clear solution. Stir phase B into phase A.

The example can be repeated with each of the polymers 2-7 according to the invention. In each case, a mouthwash with good properties is obtained.

### Example 38

### Prosthesis Adhesive

Phase A

| | |
|---|---|
| 0.20 | Bisabolol |
| 1.00 | Betacarotene |
| q.s. | Aroma oil |
| 20.00 | Cetearyl octanoate |
| 5.00 | Silicon dioxide |
| 33.80 | Mineral oil |

Phase B

| | |
|---|---|
| 1.0 | Polymer from example 1 (solid) |
| ad 100 | Dist. water |
| 35.00 | PVP (20% strength solution in water) |

Preparation:
Thoroughly mix phase A. Stir phase B into phase A.

The example can be repeated with each of the polymers 2-7 according to the invention. In each case, a prosthesis adhesive with good properties is obtained.

### Example 39

### Skinscare Cream, Type O/W

Phase A

| | |
|---|---|
| 8.00 | Cetearyl alcohol |
| 2.00 | Ceteareth-6 |
| 2.00 | Ceteareth-25 |
| 10.00 | Mineral oil |
| 5.00 | Cetearyl octanoate |
| 5.00 | Dimethicone |

Phase B

| | |
|---|---|
| 0.50 | Polymer from example 1 (solid) |
| ad 100 | Dist. water |
| 2.00 | Panthenol, propylene glycol |
| q.s. | Preservative |

Phase C

| | |
|---|---|
| q.s. | Perfume oil |

Preparation:
Heat phase A and B separately to about 80° C. Stir phase B into phase A with homogenization, briefly after homogenize. Cool to about 40° C., add phase C, homogenize again.

The example can be repeated with each of the polymers 2-7 according to the invention. In each case, a skin care cream, type O/W with good properties is obtained.

### Example 40

### Skin Care Cream, Type W/O

Phase A

| | |
|---|---|
| 6.00 | Hydrogenated castor oil PEG-7 |
| 8.00 | Cetearyl octanoate |
| 5.00 | Isopropyl myristate |
| 15.00 | Mineral oil |
| 2.00 | PEG-45/dodecyl glycol copolymer |
| 0.50 | Magnesium stearate |
| 0.50 | Aluminum stearate |

Phase B

| | |
|---|---|
| 3.00 | Glycerol |
| 0.60 | Polymer from example 1 (solid) |
| ad 100 | Dist. water |
| 0.70 | Magnesium sulfate |
| 2.00 | Panthenol |
| q.s. | Preservative |

Phase C

| | |
|---|---|
| 1.00 | Tocopherol |
| 5.00 | Tocopheryl acetate |
| q.s. | Perfume oil |

Preparation:
Heat phases A and B separately to about 80° C. Stir phase B into phase A and homogenize. Cool to about 40° C., add phase C and briefly homogenize again.

The example can be repeated with each of the polymers 2-7 according, to the invention. In each case, a skin care cream, type W/O with good properties is obtained.

### Example 41

### Lip Care Cream

Phase A

| | |
|---|---|
| 10.00 | Cetearyl octanoate |
| 5.00 | Polybutene |

Phase B

| | |
|---|---|
| 0.10 | Carbomer |

Phase C

| | |
|---|---|
| 2.00 | Ceteareth-6 |
| 2.00 | Ceteareth-25 |
| 2.00 | Glyceryl stearate |
| 2.00 | Cetyl alcohol |
| 1.00 | Dimethicone |
| 1.00 | Benzophenone-3 |
| 0.20 | Bisabolol |
| 6.00 | Mineral oil |

Phase D

| | |
|---|---|
| 1.50 | Polymer from example 1 (solid) |
| ad 100 | Dist. water |
| 3.00 | Panthenol |
| 3.00 | Propylene glycol |
| q.s. | Preservative |

Phase E

| | |
|---|---|
| 0.10 | Triethanolamine |

Phase F

| | |
|---|---|
| 0.50 | Tocopheryl acetate |
| 0.10 | Tocopherol |
| q.s. | Perfume oil |

Preparation:
Dissolve phase A to give a clear solution. Add phase B thereto and homogenize. Add phase C and melt at 80° C. Heat phase D to 80° C. Add phase D to phase ABC and homogenize. Cool to about 40° C., add phase E and phase F, homogenize again.

The example can be repeated with each of the polymers 2-7 according to the invention. In each case, a lip care cream with good properties is obtained.

### Example 42

### Shower Gel

| | |
|---|---|
| 50.00 | Sodium laureth sulfate, magnesium laureth sulfate, |
| | sodium laureth-8 sulfate, magnesium laureth-8 |
| 1.00 | Cocoamide DEA |
| 0.8 | Polymer from example 1 (solid) |
| ad 100 | Dist. water |
| 2.00 | Sodium laureth sulfate, glycol distearate, |
| | cocamide MEA, laureth-10 |
| q.s. | Preservative |
| q.s. | Perfume oil |
| 2.00 | Sodium chloride |

Preparation:
Weigh in all of the components together and stir until dissolved.

The example can be repeated with each of the polymers 2-7 according to the invention. In each case, a shower gel with good properties is obtained.

### Example 43

### Shower Gel

| | |
|---|---|
| 30.00 | Sodium laureth sulfate |
| 6.00 | Sodium cocoamphodiacetate |
| 6.00 | Cocamidopropylbetaine |
| 3.00 | Sodium laureth sulfate, glycol distearate, |
| | cocamide MEA, laureth-10 |
| 7.70 | Polyquaternium-44 |
| 0.2 | Polymer from example 1 (solid) |
| ad 100 | Dist. water |
| 1.00 | Panthenol |
| q.s. | Preservative |
| q.s. | Perfume oil |
| q.s. | Citric acid |
| 0.50 | Sodium chloride |

Preparation:
Weigh in the components of phase A and dissolve. Adjust the pH to 6 to 7.

The example can be repeated with each of the polymers 2-7 according to the invention. In each case, a shower gel with good properties is obtained.

### Example 44

### Clear Shower Gel

| | |
|---|---|
| 40.00 | Sodium laureth sulfate |
| 5.00 | Decyl glucoside |
| 5.00 | Cocamidopropylbetaine |
| 0.50 | Polyquaternium-10 |
| 2.00 | Polymer from example 1 (solid) |
| ad 100 | Dist. water |
| 1.00 | Panthenol |
| q.s. | Perfume oil |
| q.s. | Preservative |
| q.s. | Citric acid |
| 2.00 | Sodium chloride |

Preparation:
Weigh in the components of phase A and dissolve to give a clear solution.

The example can be repeated with each of the polymers 2-7 according to the invention. In each case, a clear shower gel with good properties is obtained.

### Example 45

### Shower Bath

A

| | |
|---|---|
| 40.00 | Sodium laureth sulfate |
| 5.00 | Sodium C₁₂₋₁₅ pareth-15 sulfonate |
| 5.00 | Decyl glucoside |
| q.s. | Perfume oil |
| 0.10 | Phytantriol |

B

| | |
|---|---|
| 0.1 | Guar hydroxypropyltrimonium chloride |
| 2.00 | Polymer from example 1 (solid) |
| ad 100 | Dist. water |
| 1.00 | Panthenol |
| q.s. | Preservative |
| 1.00 | Laureth-3 |
| q.s. | Citric acid |
| 2.00 | Sodium chloride |

Preparation:
Mix the components of phase A. Add the components of phase B one after the other and mix. Adjust the pH to 6 to 7.

The example can be repeated with each of the polymers 2-7 according to the invention. In each case, a shower bath with good properties is obtained.

### Example 46

### Liquid Soap

A

| | |
|---|---|
| 43.26 | Dist. water |
| 0.34 | Aminomethylpropanol |
| 3.40 | Poly(ethyl acrylate/methacrylic acid) (Luviflex ® Soft, BASF) |

B

| | |
|---|---|
| 40.00 | Sodium laureth sulfate |
| 10.00 | Cocamidopropylbetaine |
| 0.2 | Polymer from example 1 (solid) |
| ad 100 | Dist. water |
| q.s. | Perfume oil |
| q.s. | Preservative |
| 2.00 | Sodium chloride |

Preparation:
Weigh in the components of phase A and dissolve to give a clear solution. Add the components of phase B one after the other and mix.

The example can be repeated with each of the polymers 2-7 according to the invention. In each case, a liquid soap with good properties is obtained.

### Example 47

### Liquid Foot Bath

A

| | |
|---|---|
| 1.00 | Nonoxynol-14 |
| 0.10 | Bisabolol |
| 1.00 | Pine oil (Pinus Sylvestris) |

B

| | |
|---|---|
| 5.00 | PEG-8 |
| 1.50 | Polymer from example 1 (solid) |
| ad 100 | Dist. water |
| 0.50 | Triclosan |
| 30.00 | Sodium laureth sulfate |
| 3.00 | Polyquaternium-16 |
| q.s. | C.I. 19 140 + C.I. 42 051 |

Preparation:
Solubilize phase A. Mix phase B.

The example can be repeated with each of the polymers 2-7 according to the invention. In each case, a liquid foot bath with good properties is obtained.

### Example 48

### Refreshing Gel

A

| | |
|---|---|
| 0.60 | Carbomer |
| 45.40 | Dist. water |

B

| | |
|---|---|
| 0.50 | Bisabolol |
| 0.50 | Farnesol |
| q.s. | Perfume oil |
| 5.00 | PEG-40 hydrogenated castor oil |
| 0.50 | Polymer from example 1 (solid) |
| ad 100 | Dist. water |
| 1.00 | Tetrahydroxypropylethylenediamine |
| 1.50 | Menthol |
| 43.00 | Alcohol |
| q.s. | C.I. 74 180, Direct Blue 86 |

Preparation:
Allow phase A to swell. Dissolve phase B. Stir phase B into phase A.

The example can be repeated with each of the polymers 2-7 according to the invention. In each case, a refreshing gel with good properties is obtained.

### Example 49

### Roll-on Antiperspirant

A

| | |
|---|---|
| 0.40 | Hydroxyethylcellulose |
| 50.00 | Dist. water |

B

| | |
|---|---|
| 25.00 | Alcohol |
| 0.10 | Bisabolol |
| 0.30 | Farnesol |
| 2.00 | PEG-40 hydrogenated castor oil |
| q.s. | Perfume oil |

C

| | |
|---|---|
| 5.00 | Aluminum chlorohydrate |
| 3.00 | Propylene glycol |
| 3.00 | Dimethicone copolyol |
| 3.00 | Polyquaternium-16 |
| 1.50 | Polymer from example 1 (solid) |
| ad 100 | Dist. water |

Preparation:
Allow phase A to swell. Dissolve phase B and C separately. Stir phase A and B into phase C.

The example can be repeated with each of the polymers 2-7 according to the invention. In each case, a roll-on antiperspirant with good properties is obtained.

### Example 50

### Transparent Deodorant Stick

| | |
|---|---|
| 5.00 | Sodium stearate |
| 0.50 | Triclosan |
| 3.00 | Ceteareth-25 |
| 20.00 | Glycerol |
| 0.50 | Polymer from example 1 (solid) |
| ad 100 | Dist. water |
| q.s. | Perfume oil |
| 60.00 | Propylene glycol |
| 0.20 | Bisabolol |

Preparation:
Weigh phase A together, melt and homogenize. Then pour into the mold.

The example can be repeated with each of the polymers 2-7 according to the invention. In each case, a transparent deodorant stick with good properties is obtained.

### Example 51

### Water-Soluble Bath Oil

| | |
|---|---|
| 15.00 | Cetearyl octanoate |
| 15.00 | Caprylic/capric triglyceride |
| 1.00 | Panthenol, propylene glycol |
| 0.10 | Bisabolol |
| 2.00 | Tocopheryl acetate |
| 2.00 | Retinyl palmitate |
| 0.10 | Tocopherol |
| 37.00 | PEG-7 glyceryl cocoate |
| 0.4 | Polymer from example 1 (solid) |
| ad 100 | Dist. water |
| q.s. | Perfume oil |
| 23.60 | PEG-40 hydrogenated castor oil |

Preparation:
Mix and stir until everything has dissolved to give a clear solution.

The example can be repeated with each of the polymers 2-7 according to the invention. In each case, a water-soluble bath oil with good properties is obtained.

### Example 52

### Daycare Aerosol

A

| | |
|---|---|
| 4.00 | Ethylhexyl methoxycinnamate |
| 1.50 | Octocrylen |
| 9.00 | Caprylic/capric triglyceride |
| 5.00 | Simmondsia Chinensis (Jojoba) seed oil |
| 1.50 | Cyclomethicone |
| 3.00 | Hydrogenated cocoglycerides |
| 1.00 | PVP/hexadecene copolymer |
| 1.00 | Ceteareth-6, stearyl alcohol |

B

| | |
|---|---|
| 5.00 | Zinc oxide |

C

| | |
|---|---|
| 2.00 | Ceteareth-25 |
| 1.20 | Panthenol |
| 0.20 | Sodium ascorbyl phosphate |
| 0.30 | Imidazolidinylurea |
| 0.10 | Disodium EDTA |
| 1.50 | Polymer from example 1 (solid) |
| ad 100 | Dist. water |

D

| | |
|---|---|
| 0.50 | Tocopheryl acetate |
| 0.20 | Bisabolol |
| 0.33 | Caprylic/capric triglyceride, retinol |
| q.s. | Perfume oil |

Preparation:
Heat phase A to 80° C. Dissolve phase A to give a clear solution. Incorporate phase B and homogenize. Add phase C, heat to 80° C., melt and homogenize. Cool to about 40° C. with stirring, add phase D and briefly homogenize. Bottle 90% active ingredient solution: 10% propane/butane at 3.5 bar (20° C.).

The example can be repeated with each of the polymers 2-7 according to the invention. In each case, a day care aerosol with good properties is obtained.

### Example 53

### Moisturizing Cream

A

| | |
|---|---|
| 3.00 | Vitis Vinifera (Grape) seed oil |
| 1.00 | Cyclopentasiloxane, cyclohexasiloxane |
| 1.50 | Cyclomethicone |
| 2.00 | Soybean (glycine soya) oil |
| 2.00 | Ethylhexyl methoxycinnamate |
| 1.00 | Uvinul ® A Plus |
| 1.00 | Hydrogenated lecithin |
| 1.00 | Cholesterol |
| 2.00 | PEG-40 hydrogenated castor oil |
| 5.00 | Cetearyl octanoate |
| 5.00 | Caprylic/capric triglyceride |

B

| | |
|---|---|
| 3.00 | Caprylic/capric triglyceride, acrylate copolymer |

C

| | |
|---|---|
| 2.50 | Polymer from example 1 (solid) |
| ad 100 | Dist. water |
| 0.50 | Cocotrimonium methosulfate |
| 2.00 | Panthenol, propylene glycol |
| 3.00 | Glycerol |
| 0.10 | Disodium EDTA |

D

| | |
|---|---|
| 0.30 | Perfume oil |
| 0.30 | DMDM hydantoin |
| 1.00 | Tocopheryl acetate |
| 2.00 | Tocopherol |

Preparation:
Heat phase A to 80° C. Stir phase B into phase A. Heat phase C to about 80° C. and stir into phase A + B with homogenization. Cool to about 40° C. with stirring, add phase D and briefly homogenize.

The example can be repeated with each of the polymers 2-7 according to the invention. In each case, a moisturizing cream with good properties is obtained.

### Example 54

### Aerosol Hair Foam

A

| | |
|---|---|
| 2.00 | Cocotrimonium methosulfate |
| 0.20 | Perfume oil |

B

| | |
|---|---|
| 1.60 | Polymer from example 1 (solid) |
| ad 100 | Dist. water |
| 0.50 | Poly(ethyl acrylate/methacrylic acid) (Luviflex ® Soft) |
| 0.10 | Aminomethylpropanol |
| 0.20 | Ceteareth-25 |
| 0.20 | Trimethylsilylamodimethicone, trideceth-10, cetrimonium |
| | chloride |
| 0.10 | PEG-25 PABA |
| 0.20 | Hydroxyethylcellulose |
| 0.20 | PEG-8 |
| 0.20 | Panthenol |
| 15.00 | Alcohol |

C

| | |
|---|---|
| 10.00 | Propane/butane 3.5 bar (20° C.) |

Preparation:
Mix phases A and B and bottle with propellant gas.

The example can be repeated with each of the polymers 2-7 according to the invention. In each case, an aerosol hair foam with good properties is obtained.

### Example 55

### Pump Mousse

A

| | |
|---|---|
| 2.00 | Cocotrimonium methosulfate |
| q.s. | Perfume oil |

C

| | |
|---|---|
| 7.00 | Polyquaternium-46 (10% strength aqueous solution) |
| 2.50 | Polymer from example 1 (solid) |
| ad 100 | Dist. water |
| 0.50 | PEG-8 |
| 1.00 | Panthenol |
| q.s. | Preservative |
| 0.20 | PEG-25 PABA (ethoxylated p-aminobenzoic acid) |

Preparation:
Mix the components of phase A. Add the components of phase B one after the other and dissolve to give a clear solution.

The example can be repeated with each of the polymers 2-7 according to the invention. In each case, a pump mousse with good properties is obtained.

### Example 56

### Aerosol Foam

| | |
|---|---|
| 3.0 | Polymer from example 1 (solid) |
| ad 100 | Dist. water |
| 5.00 | PVP/VA copolymer (40% strength aqueous solution) |
| 0.50 | Hydroxyethylcetyldimonium phosphate |
| 0.20 | Ceteareth-25 |
| 0.40 | Perfume oil PC 910.781/Cremophor |
| q.s. | Preservative |
| 10.00 | Propane/butane 3.5 bar (20° C.) |

Preparation:
Weigh everything together, stir until dissolved, then bottle.

The example can be repeated with each of the polymers 2-7 according to the invention. In each case, an aerosol foam with good properties is obtained.

### Example 57

### Color Styling Mousse

A

| | |
|---|---|
| 2.00 | Cocotrimonium methosulfate |
| q.s. | Perfume oil |

B

| | |
|---|---|
| 6.50 | Polymer from example 1 (solid) |
| ad 100 | Dist. water |
| 0.50 | Acrylate copolymer (Luvimer ® 100 P, BASF) |
| 0.10 | Aminomethylpropanol |
| 0.20 | Ceteareth-25 |
| 0.20 | Panthenol |
| 0.20 | Hydroxyethylcellulose |
| 10.00 | Alcohol |
| 0.08 | C.I. 12245, Basic Red 76 |
| 0.05 | C.I. 42510, Basic Violet 14 |

C

| | |
|---|---|
| 10.00 | Propane/butane 3.5 bar (20° C.) |

Preparation:
Weigh everything together, stir until dissolved, then bottle. Only suitable for dark blonde and brown hair!

The example can be repeated with each of the polymers 2-7 according to the invention. In each case, a color styling mousse with good properties is obtained.

### Example 58

### Pump Hair Foam

A

| | |
|---|---|
| 1.50 | Cocotrimonium methosulfate |
| q.s. | Perfume oil |

B

| | |
|---|---|
| 2.00 | Polymer from example 1 (solid) |
| ad 100 | Dist. water |

C

| | |
|---|---|
| 0.46 | Aminomethylpropanol |
| 4.00 | PEG/PPG-25/25 dimethicone/acrylate copolymer |
| q.s. | Preservative |

Preparation:
Mix phase A. Stir phase B into phase A. Add phase C and stir until dissolved.

The example can be repeated with each of the polymers 2-7 according to the invention. In each case, a pump hair foam with good properties is obtained.

### Example 59

### Aqua Wax

| | |
|---|---|
| 10 | Polymer from example 1 (solid) |
| ad 100 | Dist. water |
| q.s. | Perfume oil |
| q.s. | Hydrogenated castor oil PEG-40 |
| 0.10 | Diethyl phthalate |
| 0.10 | Cetearyl ethylhexanoate |
| 0.10 | PEG-7 glyceryl cocoate |
| 0.10 | Preservative |
| 2.00 | Caprylic/capric triglyceride, acrylate copolymer |

Preparation:
Mix everything and homogenize. Afterstir for 15 minutes.

The example can be repeated with each of the polymers 2-7 according to the invention. In each case, an Aquawax with good properties is obtained.

### Example 60

### Rinse-Off Conditioner and Repair Treatment

A

| | |
|---|---|
| 0.20 | Cetearyl octanoate |
| 0.10 | Phytantriol |
| 2.00 | Hydrogenated castor oil PEG-40 |

B

| | |
|---|---|
| q.s. | Perfume oil |
| 2.00 | Cocotrimonium methosulfate |

C

| | |
|---|---|
| ad 100 | Dist. water |

D

| | |
|---|---|
| 2.00 | Polyquaternium-16 (20% strength aqueous solution) |
| 1.0 | Polymer from example 1 (solid) |
| 1.00 | Dimethicone copolyol |
| q.s. | Preservative |
| 10.00 | Alcohol |
| q.s. | Citric acid |

Preparation:
Mix phases A and B separately. Stir phase C into phase B.

The example can be repeated with each of the polymers 2-7 according to the invention. In each case, a rinse-off conditioner and repair treatment with good properties is obtained.

### Example 61

### Hair Treatment

A

| | |
|---|---|
| 2.00 | Ceteareth-6, stearyl alcohol |
| 1.00 | Ceteareth-25 |
| 6.00 | Cetearyl alcohol |
| 6.00 | Cetearyl octanoate |
| 0.30 | Phytantriol |

B

| | |
|---|---|
| 1.0 | Polymer from example 1 (solid) |
| ad 100 | Dist. water |
| 0.70 | Guar hydroxypropyltrimonium chloride |
| 5.00 | Propylene glycol |
| 2.00 | Panthenol |
| 0.30 | Imidazolidinylurea |

C

| | |
|---|---|
| 2.00 | Cosi Silk Soluble |
| 0.20 | Perfume |
| 0.50 | Phenoxyethanol |

Preparation:
Heat phases A and B separately to about 80° C. Homogenize phase B.

The example can be repeated with each of the polymers 2-7 according to the invention. In each case, a hair treatment with good properties is obtained.

### Example 62

### Hair Cocktail

A

| | |
|---|---|
| 0.40 | Acrylate/C₁₀₋₃₀ alkyl acrylate crosspolymer |
| 2.00 | Dimethicone |
| 3.00 | Cyclomethicone/dimethiconol |
| 2.00 | Phenyltrimethicone |
| 2.00 | Amodimethicone, cetrimonium chloride, trideceth-10 |
| 0.50 | Dimethicone copolyol |
| 1.00 | Macadamia nut oil (Ternifolia) |
| 0.50 | Tocopheryl acetate |
| 1.00 | PEG-40 hydrogenated castor oil |
| q.s. | Perfume oil |

B

| | |
|---|---|
| 0.3 | Polymer from example 1 (solid) |
| ad 100 | Dist. water |
| 0.46 | Aminomethylpropanol |
| 4.00 | PEG/PPG-25/25 dimethicone/acrylate copolymer |

Preparation:
Mix the components of phase A. Dissolve phase B. Stir phase B into phase A with homogenization.

The example can be repeated with each of the polymers 2-7 according to the invention. In each case, a hair cocktail with good properties is obtained.

### Example 63

### Permanent Wave

Wave solution
A

| | |
|---|---|
| 0.20 | Cocamidopropylbetaine |
| 0.20 | Polysorbate 20 |
| 1.55 | Polymer from example 1 (solid) |
| ad 100 | Dist. water |
| 0.20 | Disodium EDTA |
| 0.20 | Hydroxyethylcellulose |

B

| | |
|---|---|
| 8.00 | Thioglycolic acid |

C

| | |
|---|---|
| 11.00 | Ammonium hydroxide |

D

| | |
|---|---|
| 5.00 | Ammonium carbonate |

Preparation:
Weigh in the components of phase A and dissolve to give a clear solution. Stir phase B into phase A.

The example can be repeated with each of the polymers 2-7 according to the invention. In each case, a wave solution with good properties is obtained.

### Example 64

### Neutralizer

A

| | |
|---|---|
| 1.00 | PEG-40 hydrogenated castor oil |
| 0.20 | Perfume oil |
| ad 100 | Dist. water |

B

| | |
|---|---|
| 0.20 | Cocamidopropylbetaine |
| 0.20 | Ceteareth-25 |
| 2.5 | Polymer from example 1 (solid) |
| q.s. | Preservative |

C

| | |
|---|---|
| 2.30 | Hydrogen peroxide |

D

| | |
|---|---|
| q.s. | Phosphoric acid |

Preparation:
Solubilize phase A. Add the components of phase B one after the other and dissolve to give a clear solution.

The example can be repeated with each of the polymers 2-7 according to the invention. In each case, a neutralizer with good properties is obtained.

### Example 65

### Dark Brown Permanent Hair Color (Oxidation Hair Color)

A

| | |
|---|---|
| 0.20 | Sodium sulfite |
| 0.05 | Disodium EDTA |
| 0.20 | p-Phenylenediamine |
| 0.30 | Resorcinol |
| 0.20 | 4-Amino-2-hydroxytoluene |
| 0.10 | m-Aminophenol |
| 1.50 | Oleyl alcohol |
| 4.50 | Propylene glycol |
| 2.30 | Sodium C₁₂₋₁₅ pareth-15 sulfonate |
| 20.00 | Oleic acid |
| ad 100 | Dist. water |

B

| | |
|---|---|
| 1.0 | Polymer from example 1 (solid) |
| 13.70 | Ammonium hydroxide |
| 6.00 | Isopropanol |
| q.s. | Perfume |

Preparation:
Solubilize phase A. Add the components of phase B one after the other and mix. The example can be repeated with each of the polymers 2-7 according to the invention. In each case, a dark brown permanent hair color (oxidation hair color) with good properties is obtained.

### Example 66

### Developer Emulsion (pH 3-4)

| | |
|---|---|
| 3.00 | Hexadecyl alcohol |
| 1.0 | Polymer from example 1 (solid) |
| ad 100 | Dist. water |
| 1.00 | Ceteareth-20 |
| 1.00 | Sodium C₁₂₋₁₅ pareth-15 sulfonate |
| 6.00 | Hydrogen peroxide |
| 0.50 | Phosphoric acid |
| 0.01 | Acetanilide |

Preparation:
Add the components one after the other and mix.

The example can be repeated with each of the polymers 2-7 according to the invention. In each case, a developer emulsion (pH 3-4) with good properties is obtained.

### Example 67

### Pale Brown Semipermanent Hair Color

| | |
|---|---|
| 10.00 | Cocodiethanolamide |
| 4.00 | Sodium dodecylbenzylsulfonate, 50% strength |
| 1.0 | Polymer from example 1 (solid) |
| ad 100 | Dist. water |
| 6.00 | C₉₋₁₁ Pareth-3 |
| 2.50 | Sodium lauryl sulfate |
| 0.40 | 2-Nitro-p-phenylenediamine |
| 0.20 | HC Red No. 3 |
| 0.20 | HC Yellow No. 2 |

Preparation:
Add the components one after the other and mix.

The example can be repeated with each of the polymers 2-7 according to the invention. In each case, a pale brown semipermanent hair color with good properties is obtained.

### Example 68

### Shampoo

| | |
|---|---|
| 30.00 | Sodium laureth sulfate |
| 6.00 | Sodium cocoamphoacetate |
| 6.00 | Cocamidopropylbetaine |
| 3.00 | Sodium laureth sulfate, glycol distearate, cocamide |
| | MEA, laureth-10 |
| 1.0 | Polymer from example 1 (solid) |
| 2.00 | Dimethicone |
| q.s. | Perfume |
| q.s. | Preservative |
| q.s. | Citric acid |
| 1.00 | Sodium chloride |
| ad 100 | Dist. water |

Preparation:
Weigh in components and dissolve. Adjust pH to 6 to 7.

The example can be repeated with each of the polymers 2-7 according to the invention. In each case, a shampoo with good properties is obtained.

### Example 69

### Shampoo

| | |
|---|---|
| 30.00 | Sodium laureth sulfate |
| 6.00 | Sodium cocoamphoacetate |
| 6.00 | Cocamidopropylbetaine |
| 3.00 | Sodium laureth sulfate, glycol |
| | distearate, cocamide MEA, laureth-10 |
| 1.0 | Polymer from example 1 (solid) |
| 2.00 | Amodimethicone |
| q.s. | Perfume |
| q.s. | Preservative |
| q.s. | Citric acid |
| 1.00 | Sodium chloride |
| ad 100 | Dist. water |

Preparation:
Weigh in components and dissolve. Adjust pH to 6 to 7.

The example can be repeated with each of the polymers 2-7 according to the invention. In each case, a shampoo with good properties is obtained.

### Example 70

### Shampoo

| | |
|---|---|
| 40.00 | Sodium laureth sulfate |
| 10.00 | Cocamidopropylbetaine |
| 3.00 | Sodium laureth sulfate, glycol |
| | distearate, cocamide MEA, laureth-10 |
| 1.0 | Polymer from example 1 (solid) |
| 2.00 | Dow corning 3052 |
| q.s. | Perfume |
| q.s. | Preservative |
| q.s. | Citric acid |
| 2.00 | Cocamido DEA |
| ad 100 | Dist. water |

Preparation:
Weigh in components and dissolve. Adjust pH to 6 to 7.

The example can be repeated with each of the polymers 2-7 according to the invention. In each case, a shampoo with good properties is obtained.

### Example 71

### Antidandruff Shampoo

| | |
|---|---|
| 40.00 | Sodium laureth sulfate |
| 10.00 | Cocamidopropylbetaine |
| 10.00 | Disodium laureth sulfosuccinate |
| 2.50 | Sodium laureth sulfate, glycol distearate, cocamide MEA, |
| | laureth-10 |
| 1.0 | Polymer from example 1 (solid) |
| 0.50 | Climbazole |
| q.s. | Perfume |
| q.s. | Preservative |
| 0.50 | Sodium chloride |
| ad 100 | Dist. Water |

Preparation:
Weigh in components and dissolve. Adjust pH to 6 to 7.

The example can be repeated with each of the polymers 2-7 according to the invention. In each case, an antidandruff shampoo with good properties is obtained.

### Example 72

### Shampoo

| | |
|---|---|
| 25.00 | Sodium laureth sulfate |
| 5.00 | Cocamidopropylbetaine |
| 2.50 | Sodium laureth sulfate, glycol distearate, cocamide MEA, |
| | laureth-10 |
| 1.0 | Polymer from example 1 (solid) |
| q.s. | Perfume |
| q.s. | Preservative |
| 2.00 | Cocamido DEA |
| ad 100 | Dist. water |

Preparation:
Weigh in components and dissolve. Adjust pH to 6 to 7.

The example can be repeated with each of the polymers 2-7 according to the invention. In each case, a shampoo with good properties is obtained.

### Example 73

### Shampoo

| | |
|---|---|
| 20.00 | Ammonium Laureth Sulfate |
| 15.00 | Ammonium Lauryl Sulfate |
| 5.00 | Cocamidopropylbetaine |
| 2.50 | Sodium laureth sulfate, glycol distearate, cocamide |
| | MEA, laureth-10 |
| 1.0 | Polymer from example 1 (solid) |
| q.s. | Perfume |
| q.s. | Preservative |
| 0.50 | Sodium chloride |
| ad 100 | Dist. water |

Preparation:
Weigh in components and dissolve. Adjust pH to 6 to 7.
The example can be repeated with each of the polymers 2-7 according to the invention. In each case, a shampoo with good properties is obtained.

### Example 74

### Clear Shower Gel

| | |
|---|---|
| 40.00 | Sodium laureth sulfate |
| 5.00 | Decyl glucoside |
| 5.00 | Cocamidopropylbetaine |
| 1.0 | Polymer from example 1 (solid) |
| 1.00 | Panthenol |
| q.s. | Perfume |
| q.s. | Preservative |
| q.s. | Citric acid |
| 2.00 | Sodium chloride |
| ad 100 | Dist. water |

Preparation:
Weigh in components and dissolve. Adjust pH to 6 to 7.

The example can be repeated with each of the polymers 2-7 according to the invention. In each case, a clear shower gel with good properties is obtained.

### Example 75

### Shampoo

| | |
|---|---|
| 12.00 | Sodium laureth sulfate |
| 1.50 | Decyl glucoside |
| 2.50 | Cocamidopropylbetaine |
| 5.00 | Coco-glucoside glyceryl oleate |
| 2.00 | Sodium laureth sulfate, glycol distearate, cocamide MEA, |
| | laureth-10 |
| 1.0 | Polymer from example 1 (solid) |
| q.s. | Preservative |
| q.s. | Sunset Yellow C.I. 15 985 |
| q.s. | Perfume |
| 1.00 | Sodium chloride |
| ad 100 | Dist. water |

Preparation:
Weigh in components and dissolve. Adjust pH to 6 to 7.
The example can be repeated with each of the polymers 2-7 according to the invention. In each case, a shampoo with good properties is obtained.

### Example 76

### Shampoo

A

| | |
|---|---|
| 40.00 | Sodium laureth sulfate |
| 5.00 | Sodium C₁₂₋₁₅ pareth-15 sulfonate |
| 5.00 | Decyl glucoside |
| q.s. | Perfume |
| 0.10 | Phytantriol |

B

| | |
|---|---|
| 1.0 | Polymer from example 1 (solid) |
| ad 100 | Dist. water |
| 1.00 | Panthenol |
| q.s. | Preservative |
| 1.00 | Laureth-3 |
| q.s. | Citric acid |
| 2.00 | Sodium chloride |

Preparation:
Weigh in components of phase A and dissolve. Adjust pH to 6 to 7. Add phase B and mix.

The example can be repeated with each of the polymers 2-7 according to the invention. In each case, a shampoo with good properties is obtained.

1. A cosmetic preparation comprising at least one polymer A which comprises, in copolymerized form,
a) methyl methacrylate;
b) methacrylic acid;
c) acrylic acid;
d) if appropriate at least one compound with at least two free-radically polymerizable, olefinically unsaturated double bonds; and
e) if appropriate further olefinically unsaturated compounds, with the proviso that the amount by weight of component b) is at least equal to the amount by weight of component c).

2. The cosmetic preparation according to claim 1, where wherein polymer A comprises, in copolymerized form,
a) 50-85% by weight of methyl methacrylate;
b) 1-30% by weight of methacrylic acid;
c) 1-20% by weight of acrylic acid;
d) 0-5% by weight of at least one compound with at least two free-radically polymerizable, olefinically unsaturated double bonds; and
e) 0-30% by weight of further olefinically unsaturated compounds, with the proviso that, calculated as % by weight, the amount of component b) is at least equal to the amount of component c) and that the amounts of components a) to e) add up to 100% by weight.

3. The cosmetic preparation according to claim 1, wherein polymer A comprises, in copolymerized form,
a) 65-80% by weight of methyl methacrylate;
b) 5-25% by weight of methacrylic acid;
c) 3-15% by weight of acrylic acid;
d) 0-4% by weight of at least one compound with at least two free-radically polymerizable, olefinically unsaturated double bonds; and
e) 0-20% by weight of further olefinically unsaturated compounds.

4. The cosmetic preparation according to claim 1, wherein polymer A comprises, in copolymerized form,
a) 70-80% by weight of methyl methacrylate;
b) 10-20% by weight of methacrylic acid;
c) 5-10% by weight of acrylic acid;
d) 0.5-3% by weight of at least one compound with at least two free-radically polymerizable, olefinically unsaturated double bonds; and
e) 0-14.5% by weight of further olefinically unsaturated compounds.

5. The cosmetic preparation according to claim 1, wherein the weight ratio of b) to c) is at least 1.2:1.

6. The cosmetic preparation according to claim 1, wherein polymer A has a K value in the range from 25 to 45.

7. The cosmetic preparation according to claim 1, wherein the fraction of volatile organic components is at most 55% by weight, based on the cosmetic preparation.

8. The cosmetic preparation according to claim 1, wherein the preparation further has at least one cosmetically acceptable carrier B) which is selected from
i) water-miscible organic solvents;
ii) oils, fats, waxes;
iii) esters of C₆-C₃₀-monocarboxylic acids with mono-, di- or trihydric alcohols different from ii);
iv) saturated acyclic and cyclic hydrocarbons;
v) fatty acids;
vi) fatty alcohols;
vii) propellants (propellant gases); and
viii) mixtures thereof.

9. The cosmetic preparation according to claim 1 in the form of a spray product, wherein the preparation is present either in combination with a mechanical pump spray device or in combination with at least one propellant selected from the group consisting of propane, butane, dimethyl ether, fluorinated hydrocarbons and mixtures thereof.

10. A polymer A as defined in one of claim 1.

11. (canceled)

12. The cosmetic preparation according to claim 2, wherein polymer A comprises, in copolymerized form,
a) 65-80% by weight of methyl methacrylate;
b) 5-25% by weight of methacrylic acid;
c) 3-15% by weight of acrylic acid;
d) 0-4% by weight of at least one compound with at least two free-radically polymerizable, olefinically unsaturated double bonds; and
e) 0-20% by weight of further olefinically unsaturated compounds.

13. The cosmetic preparation according to claim 2, wherein polymer A comprises, in copolymerized form,
a) 70-80% by weight of methyl methacrylate;
b) 10-20% by weight of methacrylic acid;
c) 5-10% by weight of acrylic acid;
d) 0.5-3% by weight of at least one compound with at least two free-radically polymerizable, olefinically unsaturated double bonds; and
e) 0-14.5% by weight of further olefinically unsaturated compounds.

14. The cosmetic preparation according to claim 3, wherein polymer A comprises, in copolymerized form,
a) 70-80% by weight of methyl methacrylate;
b) 10-20% by weight of methacrylic acid;
c) 5-10% by weight of acrylic acid;
d) 0.5-3% by weight of at least one compound with at least two free-radically polymerizable, olefinically unsaturated double bonds; and
e) 0-14.5% by weight of further olefinically unsaturated compounds.

15. The cosmetic preparation according to claim 2, wherein the weight ratio of b) to c) is at least 1.2:1.

16. The cosmetic preparation according to claim 3, wherein the weight ratio of b) to c) is at least 1.2:1.

17. The cosmetic preparation according to claim 4, wherein the weight ratio of b) to c) is at least 1.2:1.

18. The cosmetic preparation according to claim 2, wherein polymer A has a K value in the range from 25 to 45.

19. The cosmetic preparation according to claim 3, wherein polymer A has a K value in the range from 25 to 45.

20. The cosmetic preparation according to claim 4, wherein polymer A has a K value in the range from 25 to 45.

21. The cosmetic preparation according to claim 5, wherein polymer A has a K value in the range from 25 to 45.
